(19) United States
(12) Patent Application Publication
(10) Pub. No.: US 2025/0011549 A1
(43) Pub. Date: Jan. 9, 2025

(54) A GEL COATED AIR-LIQUID-INTERFACE CULTURE SYSTEM WITH TUNABLE SUBSTRATE STIFFNESS MATCHING HEALTHY AND DISEASED LUNG TISSUES

(71) Applicant: UNIVERSITY OF VIRGINIA PATENT FOUNDATION, Charlottesville, VA (US)

(72) Inventors: Liheng CAI, Charlottesville, VA (US); Catherine CHU, Lovettsville, VA (US); Zhijian HE, Charlottesville, VA (US)

(73) Assignee: UNIVERSITY OF VIRGINIA PATENT FOUNDATION, Charlottesville, VA (US)

(21) Appl. No.: 18/623,437

(22) Filed: Apr. 1, 2024

Related U.S. Application Data
(60) Provisional application No. 63/511,901, filed on Jul. 5, 2023.

(51) Int. Cl. C08J 3/075; C08F 2/48; C08F 220/56; C08K 5/00; C12N 5/00; C12N 5/071

(52) U.S. Cl. CPC C08J 3/075; C08F 2/48; C08F 220/56; C08K 5/0025; C12N 5/0062; C12N 5/0688; C12N 2513/00; C12N 2533/54

(57) ABSTRACT

The present disclosure is directed an air-liquid-interface culture apparatus including a porous support and a polyacrylamide hydrogel layer. The porous support includes a polymer plate having a plurality of pores and a support surface. The polyacrylamide hydrogel layer is deposited onto the support surface of the porous support. The polyacrylamide hydrogel layer includes a polyacrylamide hydrogel having mechanical properties that mimic the extracellular matrix for epithelial cells in the human airway. An air-liquid-interface cell culture system disclosed herein may include the air-liquid-interface culture apparatus disclosed herein with one or more cells deposited on the polyacrylamide hydrogel layer.

### CROSS-REFERENCE TO RELATED APPLICATION

This application claims the benefit of priority to U.S. Provisional Patent Application No. 63/511,901, filed Jul. 5, 2023, and entitled “A GEL COATED AIR-LIQUID-INTERFACE CULTURE SYSTEM WITH TUNABLE SUBSTRATE STIFFNESS MATCHING HEALTHY AND DISEASED LUNG TISSUES,” the entire contents of which are incorporated by reference in the present application.

### TECHNICAL FIELD

The present disclosure is directed to culture systems for investigating and growing cells, in particular, to air-liquid interface (ALI) culture systems.

### BACKGROUND

Since its invention in the late 1980s, air-liquid-interface (ALI) culture systems have been the golden standard in vitro model for studying human airway biology and pulmonary diseases. In a conventional ALI system, however, cells are cultured on a plastic membrane, which is much stiffer than that of human airway tissue.

### SUMMARY

Therefore, an ongoing need exists for ALI culture systems that have stiffnesses that mimic human airway tissues. The present disclosure is directed to a gel-coated ALI culture system that enables the stiffness of the ALI culture system to be modified to match the stiffness of healthy airway tissues as well as unhealthy or diseased human airway tissues. Gel thicknesses that do not impair the transport of nutrients essential to cell growth are demonstrated. Similar to the conventional ALI system, the gel-ALI system of the present application allows human bronchial epithelial cells to proliferate and differentiate to a pseudostratified epithelium, capturing essential biological features in vivo. The Applicant discovered that hydrogels of different stiffness result in significant changes in morphology and migration of cells, highlighting the importance of the mechanical environment to human airway remodeling.

In a first aspect of the present disclosure, an air-liquid-interface culture apparatus includes a porous support including a polymer plate having a plurality of pores and a support surface; and a polyacrylamide hydrogel layer deposited onto the support surface of the porous support, the polyacrylamide hydrogel layer including a polyacrylamide hydrogel.

A second aspect of the present disclosure may include the first aspect, wherein the polyacrylamide hydrogel layer has a thickness of greater than or equal to 100 μm.

A third aspect of the present disclosure may include either one of the first aspect or the second aspect, wherein the polyacrylamide hydrogel layer has a Young's modulus of from 80 Pa to 21 kPa.

A fourth aspect of the present disclosure may include any one of the first through third aspects, wherein the polyacrylamide hydrogel layer has a mesh size of from 2 nm to 50 nm.

A fifth aspect of the present disclosure may include any one of the first through fourth aspects, further including one or more cells on the polyacrylamide hydrogel layer.

A sixth aspect of the present disclosure may include any one of the first through fifth aspects, further including a coating, wherein the coating is formed on an outer surface of the polyacrylamide hydrogel layer.

A seventh aspect of the present disclosure may include the sixth aspect, wherein the coating comprises photoactivable heterobifunctional crosslinker, collagen, or combinations thereof.

An eighth aspect of the present disclosure may include any one of the first through seventh aspects, wherein the porous support comprises a surface area of from 0.05% to 1% pores and an average pore size of from 0.1 μm to 10 μm.

A ninth aspect of the present disclosure may include any one of the first through eighth aspects, wherein the polyacrylamide hydrogel is a reaction product resulting from polymerization of a hydrogel precursor solution including acrylamide monomer, N,N′-methylene bis-acrylamide crosslinker, ammonium persulfate initiator, and N,N,N′,N′-tetramethylethylene diamide initiator.

A tenth aspect of the present disclosure may include any one of the first through ninth aspects, wherein the hydrogel precursor solution comprises: from 0.5 wt. % to 25 wt. % acrylamide monomer; from 0.05 wt. % to 1 wt. % N,N′-methylene bis-acrylamide crosslinker; from 0.0005 wt. % to 1 wt. % ammonium persulfate catalyst; and from 0.005 wt. % to 1 wt. % N,N,N′,N′-tetramethylethylenediamide initiator.

An eleventh aspect of the present disclosure may include a system for culturing cells, the system including: the air-liquid-interface culture apparatus of any one of the first through tenth aspects, wherein the porous support further comprises one or more support arms. The system further includes a nutrient medium, wherein the nutrient medium is held by a vessel; wherein the one or more support arms are configured to support the polymer plate above a bottom of the vessel to form a lower nutrient compartment and an upper nutrient compartment.

A twelfth aspect of the present disclosure is directed to a method of preparing an air-liquid-interface culture apparatus, the method including: preparing a polyacrylamide hydrogel precursor solution wherein the polyacrylamide hydrogel precursor solution comprises acrylamide monomer, N,N′-methylene bis-acrylamide crosslinker, ammonium persulfate initiator, and N,N,N′,N′-tetramethylethylenediamide initiator; pouring the polyacrylamide hydrogel precursor solution on to a porous surface; and polymerizing the polyacrylamide hydrogel precursor solution to form a polyacrylamide hydrogel.

A thirteenth aspect of the present disclosure may include the twelfth aspect, further including placing a coverslip over the polyacrylamide hydrogel precursor solution after pouring the polyacrylamide hydrogel precursor solution.

A fourteenth aspect of the present disclosure may include the thirteenth aspect, wherein a surface of the coverslip is hydrophobic.

A fifteenth aspect of the present disclosure may include any one of the twelfth through fourteenth aspects, wherein polymerizing the polyacrylamide hydrogel precursor solution occurs by free radical polymerization.

A sixteenth aspect of the present disclosure may include any one of the twelfth through fifteenth aspects, wherein the polyacrylamide hydrogel precursor solution comprises, based on the total weight of the polyacrylamide hydrogel precursor solution: from 0.5 wt. % to 25 wt. % acrylamide monomer; from 0.05 wt. % to 1 wt. % N,N′-methylene bis-acrylamide crosslinker; from 0.0005 wt. % to 1 wt. % ammonium persulfate catalyst; and from 0.005 wt. % to 1 wt. % N,N,N′,N′-tetramethylethylenediamide initiator.

A seventeenth aspect of the present disclosure may include any one of the twelfth through sixteenth aspects, wherein the polyacrylamide hydrogel precursor solution comprises a weight ratio of weight of the acrylamide monomer to the weight of the N,N′-methylenebisacrylamide of from 40:1 to 5:1.

An eighteenth aspect of the present disclosure may include any one of the twelfth through seventeenth aspects, further including applying sulfosuccinimidyl-6-(4′-azido-2′-nitrophenylamino) hexanoate to the polyacrylamide hydrogel to produce an activated outer surface of the polyacrylamide hydrogel; and applying collagen to the activated outer surface of the polyacrylamide hydrogel.

A nineteenth aspect of the present disclosure may be directed to a method of culturing cells, the method including: applying cells to an air-liquid-interface culture apparatus, the air-liquid-interface culture apparatus including: a porous support including a polymer plate having a plurality of pores and a support surface; and a polyacrylamide hydrogel layer deposited onto the support surface of the porous support, the polyacrylamide hydrogel layer including a polyacrylamide hydrogel; wherein the cells are applied to an upper surface of the polyacrylamide hydrogel layer; and submerging the air-liquid-interface culture apparatus in a nutrient medium, wherein the nutrient medium is present below the porous support and the nutrient medium is also present above the polyacrylamide hydrogel layer; incubating the cells until the cells reach at least 90% confluence; and removing the nutrient medium from above the polyacrylamide hydrogel layer to form an air-liquid-interface.

A twentieth aspect of the present disclosure includes the nineteenth aspect, further including activating an outer surface of the polyacrylamide hydrogel layer by applying photoactivable heterobifunctional crosslinker to the outer surface and exposing the photoactivable heterobifunctional crosslinker to ultra violet light; and applying collagen to the outer surface of the polyacrylamide hydrogel layer.

### BRIEF DESCRIPTION OF THE FIGURES

The following detailed description of specific aspects of the present disclosure can be best understood when read in conjunction with the following figures, in which like structure is indicated with like reference numerals, and in which:

FIG. 1 schematically depicts an exemplary, in vivo, cell system comprising human epithelial cells grown on soft lung extracellular matrix (ECM);

FIG. 2 schematically depicts an in vitro cell culture system, according to the prior art;

FIG. 3 schematically depicts an in vitro air-liquid-interface cell culture system comprising an air-liquid-interface culture apparatus having a polyacrylamide hydrogel layer designed to mimic an ECM, according to embodiments shown and described herein;

FIG. 4 schematically depicts polymerization of a hydrogel precursor solution to produce the polyacrylamide hydrogel layer of FIG. 3, according to embodiments shown and described herein;

FIG. 5 schematically depicts a reaction mechanism for catalysts and initiators in polymerization of the hydrogel precursor solution to produce the polyacrylamide hydrogel layer, according to embodiments shown and described herein;

FIG. 6 schematically depicts a reaction mechanism for hydrogel polymerization, according to embodiments shown and described herein;

FIG. 7 schematically depicts one embodiment of a method of preparing an air-liquid-interface culture apparatus, according to embodiments shown and described herein;

FIG. 8 schematically depicts the polyacrylamide hydrogel mesh of the polyacrylamide hydrogel layer of the in vitro air-liquid-interface culture system of FIG. 3, according to embodiments shown and described herein;

FIG. 9 schematically depicts an air-liquid-interface culture apparatus having a coating applied to an upper surface of the polyacrylamide hydrogel layer, according to embodiments shown and described herein;

FIG. 10 schematically depicts an air-liquid-interface culture system comprising the air-liquid-interface culture apparatus of FIG. 3 for an in vitro cell system, according to embodiments shown and described herein;

FIG. 11 schematically depicts an exemplary method of culturing cells using the air-liquid-interface culture apparatus of FIG. 3, according to embodiments shown and described herein;

FIG. 12 graphically depicts shear modulus (left y-axis), as a function of polyacrylamide (AAm) and N, N′-methylene bis-acrylamide (BIS) concentration (x-axis) for a polyacrylamide hydrogel layer, according to embodiments shown and described herein;

FIG. 13 schematically depicts a position in the polyacrylamide hydrogel layer relative to the porous support for measurement of the fluorescent intensity to determine permeability of the polyacrylamide hydrogel layer, according to embodiments shown and described herein;

FIG. 14A is an optical image of the fluorescently labeled polyacrylamide hydrogel layer at a start time of polymerization, according to embodiments shown and described herein;

FIG. 14B is an optical image of the fluorescently labeled polyacrylamide hydrogel layer after 1 hour following the start time of polymerization, according to embodiments shown and described herein;

FIG. 15 graphically depicts intensity (y-axis), as a function of time (x-axis) for a fluorescently labeled polyacrylamide hydrogel layer having a stiffness of 1 kPa, according to embodiments shown and described herein;

FIG. 16 graphically depicts intensity (y-axis), as a function of distance (x-axis) for a fluorescently labeled polyacrylamide hydrogel layer having a stiffness of 16 kPa, according to embodiments shown and described herein;

FIG. 17 graphically depicts intensity (y-axis), as a function of distance (x-axis) for a fluorescently labeled polyacrylamide hydrogel layer, according to embodiments shown and described herein;

FIG. 18 graphically depicts intensity (y-axis), as a function of distance (x-axis) for a fluorescently labeled polyacrylamide hydrogel layer, according to embodiments shown and described herein;

FIG. 19 graphically depicts intensity (y-axis), as a function of time (x-axis) for fluorescently labeled polyacrylamide hydrogel layers, one prepared with 10 vol. % monomer solution (AAm/BIS 10%) and the other with 20 vol. % monomer solution (AAm/BIS 20%), according to embodiments shown and described herein;

FIG. 20 graphically depicts intensity (y-axis), as a function of distance (x-axis) for fluorescently labeled polyacrylamide hydrogel layers, one prepared with 10 vol. % monomer solution (AAm/BIS 10%) and the other with 20 vol. % monomer solution (AAm/BIS 20%), at 0 minutes, 20 minutes, 40 minutes, and 60 minutes, according to embodiments shown and described herein;

FIG. 21 graphically depicts intensity (y-axis), as a function of distance (x-axis) for fluorescently labeled polyacrylamide hydrogel layers, one prepared with 10 vol. % monomer solution (AAm/BIS 10%) and the other with 20 vol. % monomer solution (AAm/BIS 20%), according to embodiments shown and described herein;

FIG. 22 schematically depicts a method of detecting antigens in cells based on the specific binding of an antibody to an antigen, according to embodiments shown and described herein;

FIG. 23 shows optical images showing ciliated cells with a tubulin marker, goblet cells with a MUC5AC marker, and nuclei with a DAPI marker on porous support with no polyacrylamide hydrogel, and two different stiffnesses of polyacrylamide hydrogel according to one or more embodiments shown and described herein, wherein the scale bar represents 300 μm and the scale bar the in insert represents 20 μm, according to embodiments shown and described herein;

FIG. 24 graphically depicts cell area (left y-axis) opposite the number of days that the cells had been incubated with an air-liquid-interface (ALI day) (x-axis), according to one or more embodiments shown and described herein;

FIG. 25 graphically depicts a goblet cell fraction (left y-axis) compared to a porous support with no polyacrylamide hydrogel, and two different stiffnesses of polyacrylamide hydrogel (x-axis), according to embodiments shown and described herein;

FIG. 26 graphically depicts a cilliated cell fraction (left y-axis) compared to porous support with no polyacrylamide hydrogel, and two different stiffnesses of polyacrylamide hydrogel (x-axis), according to embodiments shown and described herein;

FIG. 27 graphically depicts a TEER (left y-axis) as a function of ALI day (x-axis) according embodiments shown and described herein;

FIG. 28A shows confocal microscope images showing cell morphology under porous support with 10 vol. % monomer solution (AAm/BIS 10%) polyacrylamide hydrogel for ALI day 21, according to embodiments shown and described herein;

FIG. 28B shows a speed map showing cell migration under porous support with 10 vol. % monomer solution (AAm/BIS 10%) polyacrylamide hydrogel based on ALI days 7, 14, and 21, according to embodiments shown and described herein;

FIG. 29A shows confocal microscope images showing cell morphology under porous support with 20 vol. % monomer solution (AAm/BIS 20%) polyacrylamide hydrogel for ALI day 21, according to embodiments shown and described herein;

FIG. 29B shows a speed map showing cell migration under porous support with 20 vol. % monomer solution (AAm/BIS 20%) polyacrylamide hydrogel based on ALI days 7, 14, and 21, according to embodiments shown and described herein;

FIG. 30A shows confocal microscope images showing cell morphology under porous support without a polyacrylamide hydrogel for ALI day 21, according to embodiments shown and described herein;

FIG. 30B shows a speed map showing cell migration under porous support without a polyacrylamide hydrogel based on ALI days 7, 14, and 21, according to embodiments shown and described herein;

FIG. 31 graphically depicts migration speed (left y-axis) opposite the number of days that the cells had been incubated with an air-liquid-interface (ALI day) (x-axis), according to one or more embodiments shown and described herein;

FIG. 32 graphically depicts cell shape index (left y-axis) opposite the number of days that the cells had been incubated with an air-liquid-interface (ALI day) (x-axis), according to one or more embodiments shown and described herein; and

FIG. 33 graphically depicts TEER (left y-axis) opposite the gel thickness (x-axis), according to one or more embodiments shown and described herein.

Reference will now be made in greater detail to various aspects of the present disclosure, some aspects of which are illustrated in the accompanying drawings.

### DETAILED DESCRIPTION

Described herein are apparatus, systems, and methods for culturing cells. In particular, the present disclosure is directed an air-liquid-interface culture apparatus including a porous support and a polyacrylamide hydrogel layer. The porous support includes a polymer plate having a plurality of pores and a support surface. The polyacrylamide hydrogel layer is deposited onto the support surface of the porous support. The polyacrylamide hydrogel layer includes a polyacrylamide hydrogel having mechanical properties that mimic the extracellular matrix for epithelial cells in the human airway. An air-liquid-interface cell culture system disclosed herein may include the air-liquid-interface culture apparatus disclosed herein with one or more cells deposited on the polyacrylamide hydrogel layer.

The present disclosure is also directed to methods of preparing air-liquid-interface culture apparatuses including preparing a polyacrylamide hydrogel precursor solution. The polyacrylamide hydrogel precursor solution includes acrylamide monomer, N, N′-methylene bis-acrylamide crosslinker, ammonium persulfate initiator, and tetramethylethylenediamide initiator. The methods of preparing the air-liquid-interface culture apparatuses further include pouring the polyacrylamide hydrogel precursor solution onto a porous surface. The methods also further include polymerizing the polyacrylamide hydrogel precursor solution to form a polyacrylamide hydrogel.

The present disclosure is also directed to methods of culturing cells including applying cells to an air-liquid-interface culture apparatus of the present disclosure. The air-liquid-interface culture apparatus includes a porous support and a polyacrylamide hydrogel layer. The cells are applied to an upper surface of the polyacrylamide hydrogel layer. The method of culturing cells further includes submerging the air-liquid-interface culture apparatus in a nutrient medium. The nutrient medium is present below the porous support and the nutrient medium is also present above the polyacrylamide hydrogel layer. The method further includes incubating the cells until the cells reach at least 90% confluence. The method of culturing cells also further includes removing the nutrient medium from above the polyacrylamide hydrogel layer to form an air-liquid-interface.

Unlike conventional apparatus, systems, and methods for culturing cells, the presently disclosed apparatus, systems, and methods for culturing cells permit the creation on an in vitro environment that closely mimics the stiffness of the in vivo human tissues. Mimicking the stiffness of the in vivo tissues enables the cultured cells to behave more like cells in a living system, thus allowing for research that may allow more accurate study of specific diseases and better predictions for how in vivo tissues will respond to new treatments and medications. Further, the stiffness of the polyacrylamide hydrogel can be modified or fine-tuned to mimic healthy tissue or damaged tissues, such as fibrotic tissues, which are generally stiffer than healthy tissue.

Although exemplary embodiments are described in detail herein, other embodiments are contemplated. Accordingly, this disclosure is not limited in scope to the details of construction and arrangement of components described herein or illustrated in the drawings. The disclosure thus includes other embodiments and systems or methods that may be practiced or carried out in various ways.

Any of the components or modules referred to herein with regard to any of the embodiments may be integrally or separately formed with one another. Redundant functions or structures of the components or modules may be implemented or utilized. The various components may be communicated locally and/or remotely with any user/operator/customer/client or machine/system/computer/processor. The various components may be in communication via wireless and/or hardwire or other available communication means, systems and hardware. Various components and modules may be substituted with other modules or components that provide similar functions.

The systems, devices, and related components described herein may be configured to any of the various shapes along the entire continual geometric spectrum of manipulation of x, y, and z planes to provide and meet the environmental, anatomical, and structural demands and operational requirements. Locations and alignments of the various components may vary as desired or required. Various sizes, dimensions, contours, rigidity, shapes, flexibility, and materials of any of the components or portions of components in the various embodiments may be varied and utilized as desired or required. While some dimensions are provided on the aforementioned figures, the device may constitute various sizes, dimensions, contours, rigidity, shapes, flexibility, and materials as it pertains to the components or portions of components of the systems and devices, and therefore may be varied and utilized as desired or required.

As used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

In describing embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4).

As used herein, the term, “acrylamide monomer” means a vinyl-based monomer containing one acrylamide group.

As used herein, the phrase “cancer cells” refers to cells that display abnormalities in the mechanisms that regulate normal cell proliferation, differentiation, and survival.

As used herein, the term “hydrogel” means a hydrophilic or amphiphilic polymeric network composed of homopolymers or copolymers, which is insoluble due to the presence of covalent chemical crosslinks. The crosslinks provide the network structure and physical integrity. Hydrogels exhibit a thermodynamic compatibility with water, which allows them to swell in aqueous media.

As used herein, the term “medication” means a substance used for medical treatment.

As used herein, the term “nutrient” means any compound or component, whether of chemical or biological origin, which maintains or promotes the growth or proliferation of cells, or the cellular production of biologically active substances. Nutrients can include, for example, amino acids, salts, metals, sugars, lipids, nucleic acids, hormones, vitamins, fatty acids, proteins, and like substances, or combinations thereof. Other ingredients that can promote or maintain cultivation of cells in vitro can be selected by those of skill in the art, in accordance with a particular need.

As used herein, the phrase “progenitor cells” means cells that are the descendants of stem cells that can further differentiate to create specialized cell types.

As used herein, the phrase “stem cells” means precursor cells that have the capacity to self-renew and to generate multiple mature cell types.

Cell culture models allow for recapitulating essential biological features of human organs or tissues in vitro, and thus represent a powerful tool for exploring cell biology, mechanisms of diseases, drug discovery, and tissue engineering. Thus far, conventional ALI culture systems represent the gold-standard in vitro model for studying airway biology and diseases. For example, conventional ALI models are commonly used to investigate chronic respiratory diseases, including chronic obstructive pulmonary disease (COPD), asthma, and cystic fibrosis (CF).

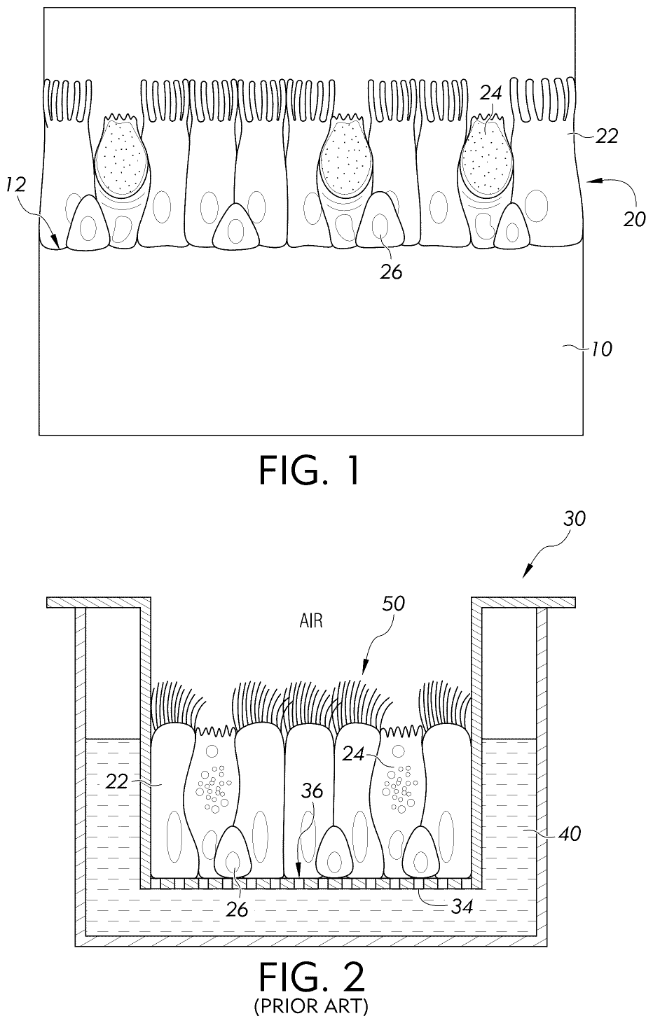

Referring now to FIG. 1, in the human airway, epithelial cells 20 are grown on the soft lung extracellular matrix (ECM) surface 12, of the ECM 10, which provides structural support and mechanical stability to the airway epithelium. The epithelial cells 20 can include ciliated cells 22, secretory cells 24 such as goblet cells, and basal cells 26. The mechanical properties of human tissue, for example the ECM 10, have been shown to influence airway cell behavior and function. See Bailey, K. E., Floren, M. L., D'Ovidio, T. J., Lammers, S. R., Stenmark, K. R., and Magin C. M. Tissue-informed engineering strategies for modeling human pulmonary diseases. Am. J. Physiol. Lung Cell. Mol. Physiol. 316 (2), L303-L320 (2019); Georges, P. C., and Janmey P. A. Cell type-specific response to growth on soft materials. J. Appl. Physiol. 98 (4), 1547-1553 (1985); and Zhou, Y., Horowitz, J. C., Naba, A., Ambalavanan, N., Atabai, K., Balestrini, J., Bitterman, P. B., Corley, R. A., Ding, B. S., Engler, A. J., Hansen, K. C., Hagood, J. S., Kheradmand, F., Lin, Q. S., Neptune, E., Niklason, L., Ortiz, L. A., Parks, W. C., Tschumperlin, D. J., White, E. S., Chapman, H. A., and Thannickal, V. J. Extracellular matrix in lung development, homeostasis and disease. Matrix Biol. 73, 77-104 (2018). For example, ECM mimicking substrate stiffness affects the morphology of alveolar epithelial cells 20 along with the size of focal adhesions and the actin cytoskeleton. See Eisenberg, J. L., Safi, A., Wei, X., Espinosa, H. D., Budinger, G. S., Takawira, D., Hopkinson, S. B., and Jones, J. C. Substrate stiffness regulates extracellular matrix deposition by alveolar epithelial cells. Research and reports in biology 2, 1-12 (2011).

Referring now to FIG. 2, a conventional ALI culture system 30 is schematically depicted. In conventional ALI culture protocols using the conventional ALI culture systems 30, a cell culture 50 comprising cells, such as but not limited to primary human bronchial epithelial (HBE) cells, are seeded onto a porous plastic support 34, such as onto a surface 36 of the porous plastic support 34. The cells in the cell culture 50 initially grow under submerged culture with culture medium 40 supplied to both apical and basal chambers. Once cells reach confluence, the system is transitioned to an ALI culture by removing the culture medium 40 at the apical compartment (i.e., compartment above the plastic support 34). Transitioning the conventional ALI culture system 30 to the ALI culture initiates cells to differentiate into different types of cells. For example, HBE cells 20 differentiate into ciliated cells 22, goblet cells (secretory cells 24), and basal cells 26, and eventually form a pseudostratified columnar epithelium, thus recapitulating important biological features of the human airway surface in vivo.

A major limitation of conventional ALI culture systems 30 is that the cells 20 are grown on a porous plastic support 34, which is made of stiff, fragile plastics (e.g., polyester or polyethylene terephthalate (PET)). These stiff, fragile plastics can have stiffness on the order of gigapascals (GPa), which is more than 105 times stiffer than some human tissues, for example, human airway tissues.

Another drawback of conventional ALI culture systems 30 is that the plastic membrane of the porous plastic support 34 has a fixed thickness and cannot be adjusted. However, the stiffness of various tissues can change with certain diseases or injuries. For example, the lung ECM varies with pulmonary injuries or diseases. For instance, idiopathic pulmonary fibrosis (IPF) is a progressive lung disease of unknown cause that is associated with dysregulated lung remodeling that produces massive ECM deposition in the lung, leading to tremendously high lung stiffness compared to healthy lung tissue.

A variety of materials have been used to substitute or coat the plastic membrane of the porous plastic support 34 in the conventional ALI culture systems 30. Naturally derived materials, such as collagen, alginate, elastin, and reconstituted basement membrane derived from extracts of Engelbreth-Holm-Swarm mouse tumors (commercially available from Corning Life Sciences as “Matrigel”), can better mimic the biophysical and biochemical properties of various tissues. By contrast, synthetic materials can precisely control the stiffness. For example, polydimethylsiloxane (PDMS) is commonly used as a porous membrane due to its high elasticity, opacity, and biocompatibility. Although stretchable PDMS membranes have been often integrated into microfluidic devices to mimic in vivo conditions such as the cyclic mechanical strain of the lung alveolar barrier, PDMS absorbs hydrophobic small molecules and drugs, which may affect the results of drug testing.

Another synthetic material is polyacrylamide (PAAm) hydrogel. A magnetic microboat device integrating with PAAm hydrogel has been developed to mimic the ECM of the healthy lung and the lung with cystic fibrosis. See Chandrasekaran, A., Kouthouridis, S., Lec, W., Lin, N., Ma, Z., Turner, M. J., Hanrahan, J. W., and Moraes, C. Magnetic microboats for floating, stiffness tunable, air-liquid interface epithelial cultures. Lab. Chip 19, 2786-2798 (2019). However, the PAAm gel thickness of the magnetic microboat was 1 mm thick, prolonging the time for nutrients and medications to transport through the PAAm gel. Additionally, the magnetic microboat required manual manipulation of magnetic fields.

Therefore, there is an ongoing need for in vitro cell culture apparatus, systems, and methods that provide adjustable stiffness that mimics in vivo stiffness and that provide improved nutrient and drug adsorption. The present disclosure meets these and other needs by providing apparatus, systems, and methods for air-liquid-interface cultures that include a porous support and a polyacrylamide hydrogel layer deposited onto a support surface of the porous support.

The air-liquid-interface culture apparatuses and systems according to embodiments described herein allow the stiffness of the culture system to be controlled so that the culture system mimics the stiffness of either healthy or diseased tissues. Specifically, the stiffness of the hydrogel layer may be varied by adjusting the concentration of the monomer, the crosslinker, or both. Additionally, the air-liquid-interface culture apparatuses and systems according to embodiments described herein may allow for efficient diffusion of nutrients and medications through the hydrogel layer. This efficient diffusion of nutrients and medications through the hydrogel layer may allow for better modeling of treatment and drug response. Furthermore, the air-liquid-interface culture apparatuses and systems according to embodiments described herein may require less manual manipulation than culture systems requiring magnetic fields.

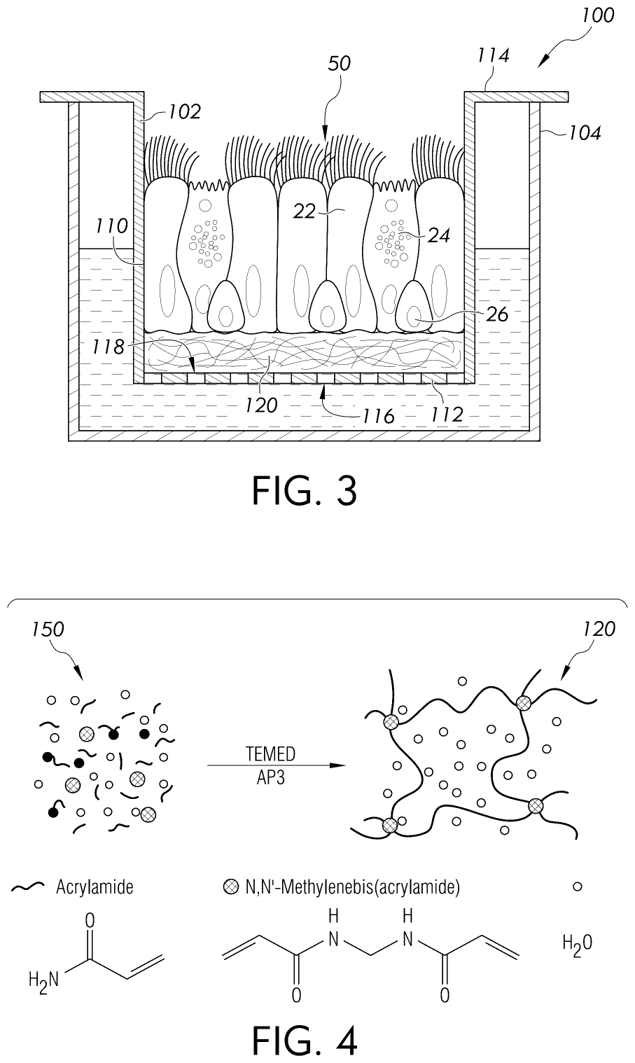

Referring now to FIG. 3, one embodiment of the air-liquid-interface culture system 100 of the present disclosure comprising an air-liquid-interface culture apparatus 102 positioned in a well 104 of a culture plate or microplate (e.g., titer plate) is schematically depicted. The air-liquid-interface culture apparatuses 102 of the present disclosure includes a porous support 110 having a porous polymer plate 112 and one or more support arms 114 configured to support the porous polymer plate 112 within the well 104. The porous polymer plate 112 has a plurality of pores 116 and a support surface 118. The air-liquid-interface culture apparatuses 102 further include a polyacrylamide hydrogel layer 120 that is deposited onto the support surface 118 of the porous support 110. The polyacrylamide hydrogel layer 120 includes a polyacrylamide hydrogel.

The porous support 110 of the present disclosure includes the porous polymer plate 112 having the plurality of pores 116 extending through the porous polymer plate 112. The polymer plate 112 may be constructed of a polymer material that does not readily absorb nutrients, medications, or other small molecules. In embodiments, the porous polymer plate 112 may be formed from glass or plastics, such as but not limited to polyester, polyethylene terephthalate, or combinations thereof. In embodiments, the polymer plate is a single member. In embodiments, the porous polymer plate 112 may include two or more members. The porous polymer plate 112 may be neutral in charge so that the porous polymer plate 112 does not interfere with the diffusion of charged molecules through the pores 116 of the porous polymer plate 112.

In embodiments, the porous support 110 may include a pore surface area that is from 0.05% to 1% of the total surface area of the support surface 118 of the porous polymer plate 112, where the pore surface area is a sum of the cross-sectional areas of all the pores 116 in the porous polymer plate 112. The total surface area of the support surface 118 is the total area within an outer perimeter of the support surface 118, including the pore surface area of the pore openings. In embodiments, the porous polymer plate 112 of the porous support 110 may have a pore surface area that is from 0.05% to 1%, from 0.1% to 0.9%, from 0.2% to 0.8%, from 0.3% to 0.7%, or from 0.4% to 0.6% of the total surface area of the support surface 118. The porous polymer plate 112 of the porous support 110 may have an average pore size of from 0.1 μm to 10 μm. In embodiments, the porous polymer plate 112 of the porous support 110 may have an average pore size of from 0.1 μm to 10 μm, from 0.2 μm to 8 μm, from 0.3 μm to 5 μm, from 0.4 μm to 3 μm, or from 0.4 to 1 μm.

Referring again to FIG. 3, the air-liquid-interface culture apparatuses 102 also includes the polyacrylamide hydrogel layer 120 deposited onto the support surface 118 of the porous support 110. The polyacrylamide hydrogel layer 120 comprises a polyacrylamide hydrogel. The stiffness of the polyacrylamide hydrogel layer 120 can be controlled to mimic the stiffness of tissue in healthy or diseased in vivo systems. Additionally, the polyacrylamide hydrogel layer 120 may permit the diffusion of nutrients and other biologically important molecules.

Methods of preparing the polyacrylamide hydrogel layer 120 may include mixing acrylamide, a crosslinker, and a solvent to form a hydrogel precursor solution. The solvent may include deionized water. In embodiments, the acrylamide and the crosslinker may be prepared as separate solutions before mixing the acrylamide, the crosslinker, and the solvent. Referring now to FIG. 4, the hydrogel precursor solution 150 may then be polymerized to form the polyacrylamide hydrogel layer 120. To catalyze polymerization, a catalyst may be added to the hydrogel precursor solution. In embodiments, the catalyst is in solution. To initiate polymerization, an initiator may be added to the hydrogel precursor solution. In embodiments, the initiator is in solution.

The acrylamide may include acrylamide monomer. The hydrogel precursor solution may include from 0.5 wt. % to 25 wt. % acrylamide monomer, based on the total weight of the hydrogel precursor solution. In embodiments, the hydrogel precursor solution may include from 0.5 wt. % to 25 wt. %, from 1 wt. % to 15 wt. %, or from 5 wt. % to 10 wt. % acrylamide monomer, based on the total weight of the hydrogel precursor solution.

The crosslinker may include N,N′-methylene bis-acrylamide crosslinker (BIS). The hydrogel precursor solution may include from 0.05 wt. % to 1 wt. % N,N′-methylene bis-acrylamide as the crosslinker, based on the total weight of the hydrogel precursor solution. In embodiments, the hydrogel precursor solution may include from 0.05 wt. % to 1 wt. %, from 0.07 wt. % to from 0.7 wt. %, or from 0.1 wt. % to 0.5 wt. % N,N′-methylene bis-acrylamide as the crosslinker, based on the total weight of the hydrogel precursor solution.

The catalyst may include ammonium persulfate (APS). The hydrogel precursor solution may include from 0.0005 wt. % to 1 wt. % ammonium persulfate catalyst, based on the total weight of the hydrogel precursor solution. In embodiments, the hydrogel precursor solution may include from 0.0005 wt. % to 1 wt. %, from 0.001 wt. % to 0.5 wt. %, or from 0.01 wt. % to 0.1 wt. % ammonium persulfate catalyst, based on the total weight of the hydrogel precursor solution.

The initiator may include N,N,N′, N′-tetramethylethylenediamine (TEMED). The hydrogel precursor solution may include from 0.005 wt. % to 1 wt. % N,N,N′,N′-tetramethylethylenediamide initiator, based on the total weight of the hydrogel precursor solution. In embodiments, the hydrogel precursor solution may include from 0.005 wt. % to 1 wt. %, from 0.01 wt. % to 0.5 wt. %, or from 0.05 wt. % to 0.1 wt. % N,N,N′,N′-tetramethylethylenediamide initiator, based on the total weight of the hydrogel precursor solution.

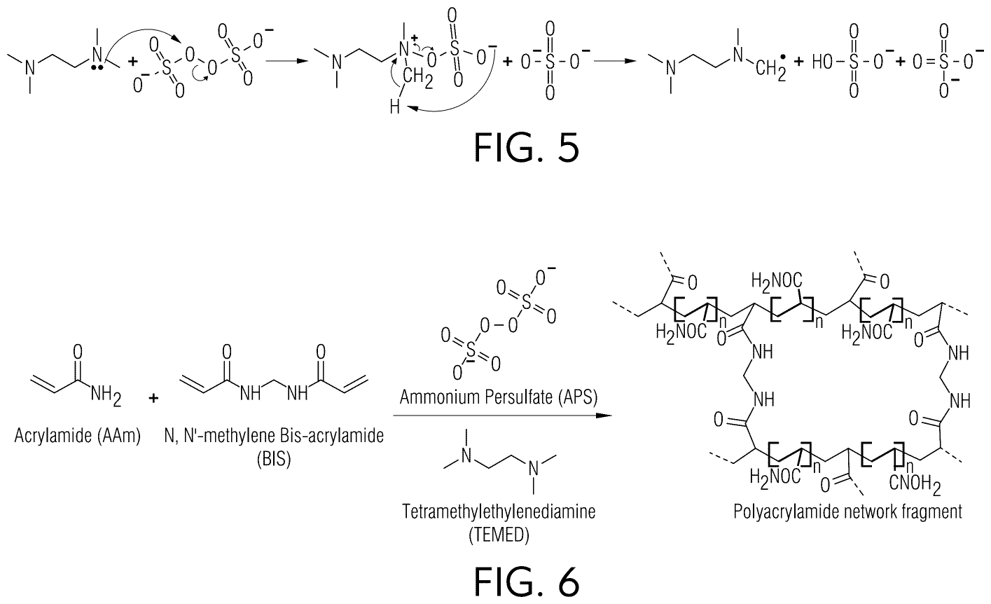

In embodiments, APS may be used as a catalyst and TEMED may be used as an initiator of the polymerization reaction. When APS is combined with TEMED, the lone pair of electrons of the nitrogen atoms in TEMED attack the oxygen atoms of the oxygen-oxygen (O—O) bond of the APS to form a nitrogen-oxygen (N—O) bond and break the oxygen-oxygen (O—O) bond in the APS, as shown in FIG. 5. This creates a TEMED-sulfate intermediate and a sulfate ion. Then the oxygen anion of the TEMED-sulfate intermediate extracts a hydrogen from the dimethyl group of the TEMED-sulfate intermediate to form a TEMED radical ion and another radical sulfate ion . . .

Consequently, the free radicals begin the polymerization by breaking the double bonds (vinyl groups) of the acrylamide and converting acrylamide monomers into free radicals, as shown in FIG. 6. Then, the acrylamide radicals react with inactivated monomers to start the polymerization chain reaction. The polymer chains are randomly crosslinked with bis-acrylamide and eventually form the polyacrylamide hydrogel of the polyacrylamide hydrogel layer. FIG. 6 shows a fragment of the polyacrylamide network making up the polyacrylamide hydrogel layer.

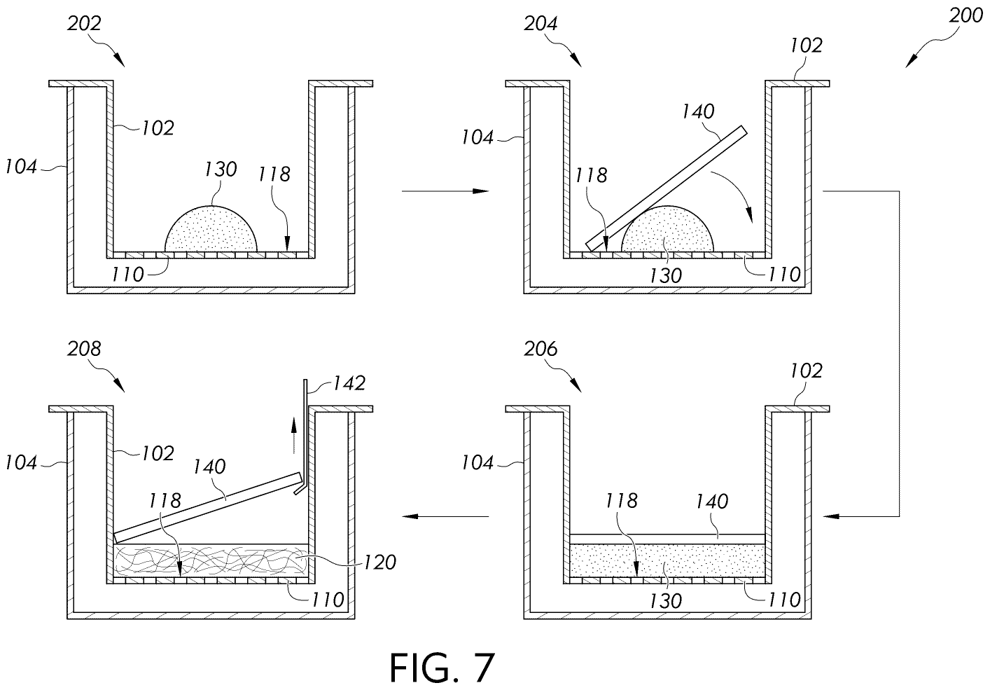

Referring now to FIG. 7, one embodiment of the methods 200 of forming the polyacrylamide hydrogel layer 120 onto the support surface 118 of the porous support 110 to form the air-liquid-interface culture apparatus 102 is schematically depicted. The porous support 110 may be positioned within the well 104 of the culture plate or microplate apparatus. The methods 200 may include transferring a volume of the hydrogel precursor solution 130 to the support surface 118 in step 202. In embodiments, the volume of the hydrogel precursor solution 130 transferred to the support surface 118 may influence the resulting thickness of the polyacrylamide hydrogel layer 120 formed on the support surface 118. The methods 200 may further include covering the hydrogel precursor solution 130 and support surface 118 with a cover 140, such as a coverslip, as in step 204. The cover 140 may reduce or prevent the hydrogel solution 130 from reacting with oxygen from the air, as oxygen may impair free radical polymerization. In embodiments, the hydrogel precursor solution 130 may be spread on the support surface 118. In embodiments, the cover 140 may be used to spread the hydrogel precursor solution 130 on the support surface 118.

After depositing the hydrogel precursor solution 130 on to the support surface 118 and covering the hydrogel precursor solution 130 and the support surface 118 with the cover 140, the methods may include polymerizing the hydrogel precursor solution 130, as in step 206 of FIG. 7. Polymerization may be passive, meaning that the hydrogel precursor solution 130 may be allowed to polymerize without taking any further action to propagate the polymerization reaction. In embodiments, the hydrogel precursor solution 130 may be allowed to polymerize for 30 minutes. In embodiments, the hydrogel precursor solution 130 may be allowed to polymerize for from 20 minutes to 2 hours, from 20 minutes to 90 minutes, from 20 minutes to 60 minutes, from 20 to 45 minutes, from 20 minutes to 30 minutes, from 30 minutes to 2 hours, from 30 minutes to 90 minutes, from 30 minutes to 60 minutes, from 30 to 45 minutes, from 45 minutes to 2 hours, from 45 minutes to 90 minutes, from 45 minutes to 60 minutes, from 60 minutes to 2 hours, from 60 minutes to 90 minutes, or from 90 minutes to 2 hours.

In embodiments, after the hydrogel precursor solution 130 has been allowed to polymerize to produce the polyacrylamide hydrogel layer 120, the air-liquid-interface culture apparatus 102 may be sealed and stored for later use. In embodiments, the air-liquid-interface culture apparatus 102 may be sealed with parafilm. In embodiments, the air-liquid-interface culture apparatus 102 may be stored at a storage temperature of from 1° C. to 25° C., such as at about 4° C. In embodiments, the method may include removing the cover 140 from the polyacrylamide hydrogel layer 120, as in step 208 of FIG. 7. Removing the cover 140 may include lifting the cover 140 off the polyacrylamide hydrogel layer 120 with a cover removal tool 142.

In embodiments, the polyacrylamide hydrogel layer 120 may have a stiffness of from 0.25 kPa to 50 kPa, as determined according to the rheological characterization of the polyacrylamide hydrogel, explained in detail below. In embodiments, the polyacrylamide hydrogel layer 120 may have a stiffness of from 0.25 kPa to 50 kPa, from 0.5 kPa to 40 kPa, from 1 kPa to 25 kPa, or from 1.5 kPa to 15 kPa.

In embodiments, the polyacrylamide hydrogel layer may have a shear modulus equal to shear stress divided by shear strain, which are determined according to the test methods discussed herein. The shear modulus of the polyacrylamide hydrogel layer may be from 0.01 kPa to 35 kPa, as determined according to the rheological characterization of the polyacrylamide hydrogel test method, explained in greater detail below. In embodiments, the shear modulus of the polyacrylamide hydrogel layer may be from 0.01 kPa to 35 kPa, from 0.05 kPa to 30 kPa, from 0.1 kPa to 25 kPa, or from 0.5 kPa to 20 kPa, as determined according to the test methods disclosed herein.

In embodiments, the polyacrylamide hydrogel layer has a Young's modulus as defined as Equation (1), wherein E is the Young's modulus, G is the shear modulus, and v is the Poisson's ratio. For the polyacrylamide hydrogel layer, a Poisson's ratio of 0.5 is used in Equation (1).

$$E = 2 G ( 1 + v ) \qquad Equation ( 1 )$$

The Young's modulus of the polyacrylamide hydrogel layer may be from 0.03 kPa to 105 kPa, as determined from the shear modulus according to Equation (1). In embodiments, the Young's modulus of the polyacrylamide hydrogel layer may be from 0.03 kPa to 105 kPa, from 0.15 kPa to 90 kPa, from 0.3 kPa to 75 kPa, or from 1.5 kPa to 60 kPa. In embodiments, the polyacrylamide hydrogel layer may have a Young's modulus of from 0.5 to 5 kPa, similar to that of healthy lung tissue. In embodiments, the polyacrylamide hydrogel layer may have a Young's modulus of from 15 kPa to 100 kPa, similar to that of diseased lung tissue.

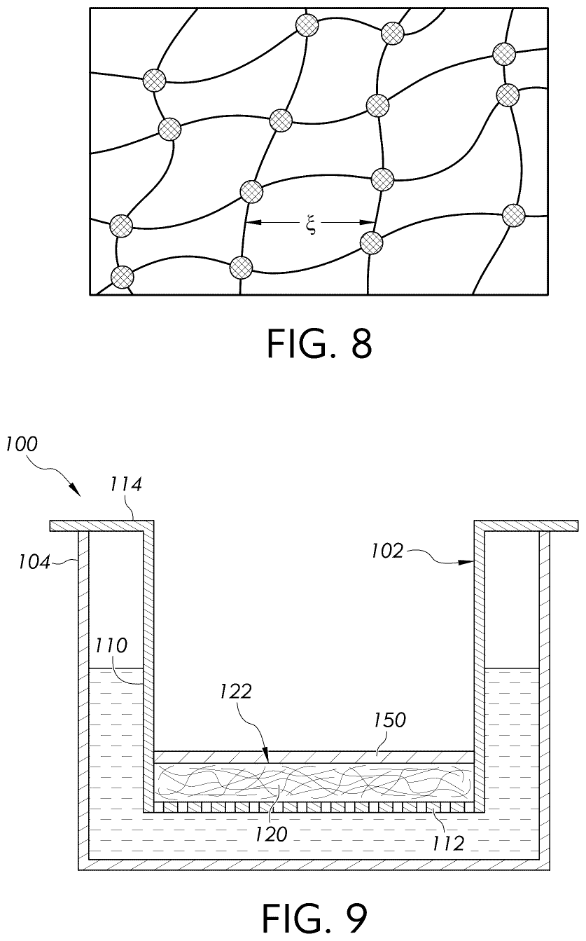

Referring now to FIG. 8, in embodiments, the polyacrylamide hydrogel layer 120 includes long polymer chains that are chemically crosslinked to create a three dimensional structure of a polymer network, which forms a mesh, as shown in FIG. 8. The mesh allows substances smaller than the size of the mesh to pass through the polyacrylamide hydrogel layer 120. The polyacrylamide hydrogel layer 120 has a mesh size (ξ) that is an average linear distance between two adjacent cross-linked long polymer chains. The mesh size (ξ) may be determined using Equation (2), wherein where G is the shear modulus as determined according to the test methods discussed herein, k_{B} is the Boltzmann constant, and T is the absolute temperature, as.

$$ξ \approx {( \frac{{k}_{B} T}{G} )}^{\frac{1}{3}} \qquad Equation ( 2 )$$

The mesh size (ξ) of the polyacrylamide hydrogel layer may be from 1 nm to 50 nm. In embodiments, the mesh size (ξ) of the polyacrylamide hydrogel layer may be from 1 nm to 50 nm, from 2 nm to 40 nm, from 3 nm to 30 nm, from 4 nm to 20 nm, or from 5 nm to 18 nm. For example, the nutrient medium, described in further detail below, may include albumin, which is a protein having a radius of about 6.4 nm. Thus, the mesh size of the polyacrylamide hydrogel layer 120 may allow the diffusion of albumin and other large nutrients through the polymer network of the polyacrylamide hydrogel layer 120.

In embodiments, the polyacrylamide hydrogel layer 120 may have a thickness of at least 100 μm. In embodiments, the polyacrylamide hydrogel layer 120 may have a thickness of from 100 μm to 1000 μm, from 100 μm to 500 μm, or from 100 μm to 250 μm.

The physical properties of the polyacrylamide hydrogel layer 120 may be modified to mimic different tissue environments. In embodiments, the physical properties of the polyacrylamide hydrogel layer 120 may be modified by adjusting the weight percentage of acrylamide and crosslinker in the polyacrylamide hydrogel precursor solution 130. A greater weight percentage of both acrylamide and crosslinker may increase both the shear modulus and the Young's modulus of the polyacrylamide hydrogel layer 120.

In embodiments, methods of preparing the air-liquid-interface culture apparatus 102 may include modifying the stiffness, shear modulus, Young's modulus, or combinations thereof of the polyacrylamide hydrogel layer 120 by increasing or decreasing a concentration of the acrylamide monomer in the polyacrylamide hydrogel precursor solution. In embodiments, the stiffness, shear modulus, Young's modulus, or combinations thereof of the polyacrylamide hydrogel layer 120 may be modified to mimic the stiffness, shear modulus, Young's modulus, or combinations thereof, of healthy lung tissue. In embodiments, the stiffness, shear modulus, Young's modulus, or combinations thereof of the polyacrylamide hydrogel layer 120 may be modified to mimic the stiffness, shear modulus, Young's modulus, or combinations thereof, of non-healthy or diseased lung tissue.

Referring now to FIG. 9, in embodiments, the air-liquid-interface culture apparatus 102 may include a coating 150, wherein the coating 150 is formed on an outer surface 122 of the polyacrylamide hydrogel layer 120. The coating 150 may include a photoactivatable heterobifunctional crosslinker, collagen, or combinations thereof. In embodiments, the photoactivatable heterobifunctional crosslinker may include sulfosuccinimidyl-6-(4′-azido-2′-nitrophenylamino) hexanoate. In embodiments, the collagen may include type I collagen. The photoactivatable heterobifunctional crosslinker may be activated to bind to the polyacrylamide hydrogel layer 120.

Activating the photoactivatable heterobifunctional crosslinker may include exposing the photoactivatable heterobifunctional crosslinker to ultraviolet (UV) light. In embodiments, the photoactivatable heterobifunctional crosslinker may be exposed to UV light for from 1 second to 1 hour, from 1 minute to 30 minutes, or from 2 minutes to 10 minutes. The UV light may have a wavelength of from 100 nm to 400 nm. The photoactivatable heterobifunctional crosslinker may also bind to the collagen, thereby linking the collagen and the polyacrylamide hydrogel layer 120. After activating the photoactivatable heterobifunctional crosslinker, the air-liquid-interface culture apparatus 102 may be rinsed to remove any unreacted photoactivatable heterobifunctional crosslinker. The air-liquid-interface culture apparatus 102 may be stored at a storage temperature of from 1° C. to 25° C., such as at about 4° C. After storage, the air-liquid-interface culture apparatus 102 may be rinsed again, and the air-liquid-interface culture apparatus 102 may be sterilized. In embodiments, the air-liquid-interface culture apparatus 102 may be sterilized by exposing the air-liquid-interface culture apparatus 102 to UV light for from 1 minute to 24 hours, from 5 minutes to 12 hours, from 10 minutes to 6 hours, or from 15 minutes to 2 hours.

In embodiments, the methods of preparing the air-liquid-interface culture apparatus 102 may include applying photoactivatable heterobifunctional crosslinker and collagen to the outer surface 122 of the polyacrylamide hydrogel layer 120 and exposing the photoactivatable heterobifunctional crosslinker to UV light. Methods of preparing an air-liquid-interface culture apparatus 102 may include first applying photoactivatable heterobifunctional crosslinker to the outer surface of the polyacrylamide hydrogel layer 120 and exposing the photoactivatable heterobifunctional crosslinker to UV light and then applying collagen to the outer surface of the polyacrylamide hydrogel layer 120. Methods of preparing an air-liquid-interface culture apparatus 102 may include rinsing the air-liquid-interface culture apparatus 102 to remove unreacted photoactivatable heterobifunctional crosslinker. Methods of preparing an air-liquid-interface culture apparatus 102 may include storing the air-liquid-interface culture apparatus 102 at a storage temperature of from 1° C. to 25° C., such as at about 4° C. Methods of preparing an air-liquid-interface culture apparatus 102 may further include sterilizing the air-liquid-interface culture apparatus 102 by exposing the air-liquid-interface culture apparatus 102 to UV light for an exposure time of from 1 minute to 24 hours, from 5 minutes to 12 hours, from 10 minutes to 6 hours, or from 15 minutes to 2 hours.

In embodiments, the air-liquid-interface culture apparatus 102 may further include one or more cells on the polyacrylamide hydrogel layer 120, on the coating 150, or within the coating 150. In embodiments, the one or more cells may be deposited on the outer surface 122 of the polyacrylamide hydrogel layer 120. The cells may be applied by seeding the cells on the polyacrylamide hydrogel layer 120 or on the coating 150. The coating 150 may be formed by the photoactivatable heterobifunctional crosslinker, collagen, or combinations thereof after the photoactivatable heterobifunctional crosslinker, collagen, or combinations thereof have been applied to the polyacrylamide hydrogel layer 120. Seeding the cells on the polyacrylamide hydrogel layer 120 or the coating 150 may include spreading an amount of cells onto the polyacrylamide hydrogel layer 120 or the coating 150. In embodiments, one or more cells on the polyacrylamide hydrogel layer 120 or the coating 150 may be stem cells or progenitor cells. In embodiments, the one or more cells may be cancer cells. In embodiments, the cells may be basal cells.

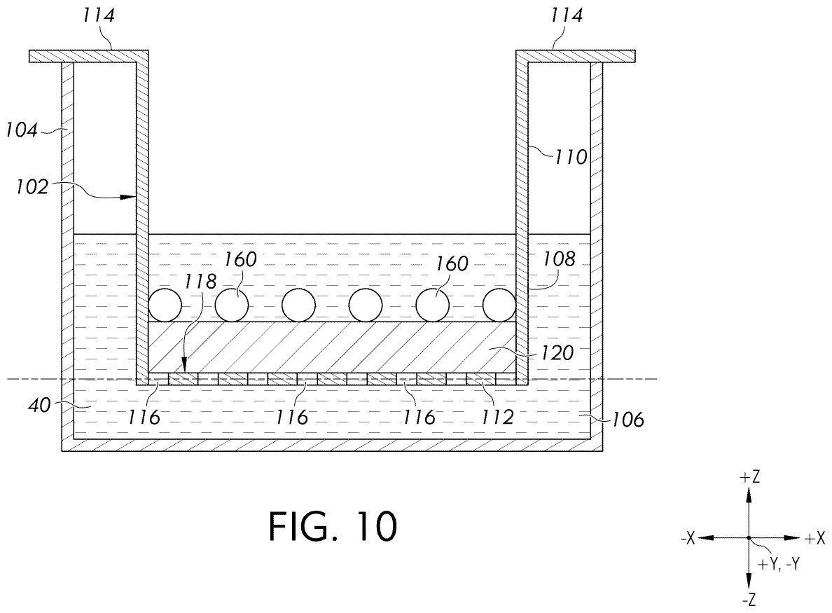

Referring now to FIG. 10, one embodiment of the air-liquid-interface culture system 100 for culturing cells is schematically depicted. The air-liquid-interface culture system 100 for culturing cells includes the air-liquid-interface culture apparatus 102, as previously described herein, and the well 104. The porous support 110 may comprise one or more of the support arms. The nutrient medium is held by a vessel. The one or more support arms 114 may be configured to support the porous polymer plate 112 above a bottom of the vessel or well 104 to form a lower nutrient compartment 106 and an upper nutrient compartment 108.

Referring again to FIG. 10, the air-liquid-interface culture system 100 may include a culture medium 40 disposed within the well 104 or vessel. The well 104 may include a side wall and the bottom of the well 104. The bottom and sidewall of the well 104 may define a volume capable of containing the nutrient medium 40. The well 104 may be shaped so that nutrient medium 40 may be added or removed from the well 104 without the nutrient medium 40 passing through the layer of cells on the polyacrylamide hydrogel layer 120 in a downward direction (i.e., the −Z direction of the coordinate axis in FIG. 10).

The nutrient medium 40 may include glycoproteins, vitamins, inorganic salts, growth factors, hormones, antibiotics, trace elements that provides nutrients to the cultured cells, or combinations of these. The nutrient medium 40 may include any other substances for cell growth and/or study of cell behaviors.

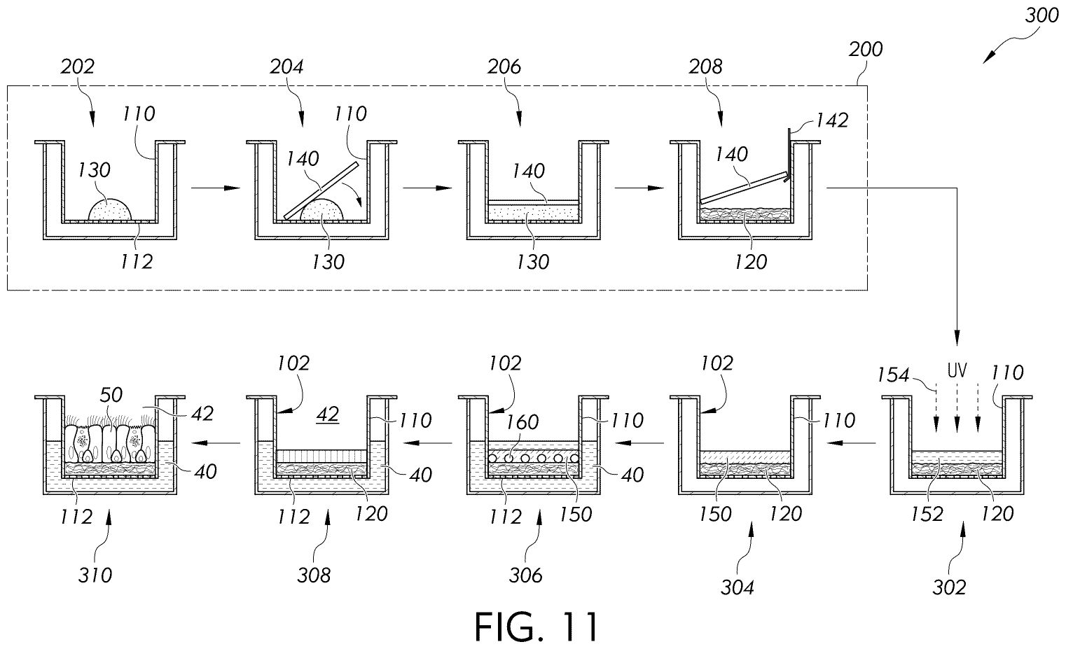

Referring now to FIG. 11, one embodiment of a method 300 of culturing cells using the air-liquid-interface culture apparatus 100 is schematically depicted. The method 300 of culturing cells may include the method 200 of preparing the air-liquid-interface culture apparatus 100, as previously described in relation to FIG. 7. In particular, the method 300 may include preparing the polyacrylamide hydrogel precursor solution 130 wherein the polyacrylamide hydrogel precursor solution comprises acrylamide monomer, N,N′-methylene bis-acrylamide crosslinker, ammonium persulfate initiator, and N,N,N′,N′-tetramethylethylenediamide initiator. The method 300 may further include pouring the polyacrylamide hydrogel precursor solution onto the support surface 118 of the porous support 110, as indicated in step 202. The method 300 may further include placing a cover 140, such as a coverslip, over the polyacrylamide hydrogel precursor solution 130, as shown in step 204, after pouring the polyacrylamide hydrogel precursor solution 130 onto the support surface 118 of the porous support 110. The method 300 may further comprise polymerizing the polyacrylamide hydrogel precursor solution 130 to form a polyacrylamide hydrogel layer 120, as in step 206. After polymerization, the method 300 may include removing the cover 140 from the polyacrylamide hydrogel layer 120.

Following formation of the polyacrylamide hydrogel layer 120, the method 300 may further include adding the coating 150 onto the polyacrylamide hydrogel layer 120. In embodiments, the method 300 may further include adding the photoactivatable heterobifunctional crosslinker to the surface of the polyacrylamide hydrogel layer and exposing the photoactivatable heterobifunctional crosslinker 152 to UV light 154, as indicated in step 302 of method 300. As previously discussed, adding the photoactivatable heterobifunctional crosslinker 152 to the polyacrylamide layer 120 and exposing the photoactivatable heterobifunctional crosslinker 152 to UV light may activate the outer surface of the polyacrylamide hydrogel layer 120 to bond collagen to the upper surface of the polyacrylamide hydrogel layer 120. The methods 300 may further include applying a coating layer 150 comprising collagen onto the activated outer surface of the polyacrylamide hydrogel layer 120, as shown in step 304. In embodiments, the methods 300 of culturing cells may further include activating the outer surface of the polyacrylamide hydrogel layer 120 by applying the photoactivatable heterobifunctional crosslinker to the outer surface and exposing the photoactivatable heterobifunctional crosslinker to UV light. The methods 300 of culturing cells may also further include applying collagen to the outer surface of the polyacrylamide hydrogel layer to form the coating 150 thereon.

Still referring to FIG. 11, the methods 300 of culturing cells include applying cells 160 to the air-liquid-interface culture apparatus 102, as described herein above, wherein the cells 160 are applied to the outer surface of the polyacrylamide hydrogel layer 120, as shown in step 306 of FIG. 11. The methods may further include submerging the air-liquid-interface culture apparatus 102 in a nutrient medium 40, wherein the nutrient medium 40 is present below the support surface 112 of the porous support 110 and the nutrient medium 40 is also present above the polyacrylamide hydrogel layer 120, as shown in step 306. The methods 300 may further include incubating the cells 160 until the cells reach at least 90% confluence. Once the cells 160 reach at least 90% confluence or greater, the methods 300 may further include removing the nutrient medium 40 from above the polyacrylamide hydrogel layer 120 to form an air-liquid-interface, as shown in step 308 of FIG. 11.

### Test Methods

### Polyacrylamide Hydrogel Thickness Determination.

To quantify the polyacrylamide hydrogel thickness and permeability, fluorescein isothiocyanate-labeled dextran (Texas Red, MW 70,000 Da) was added to the hydrogel precursor solution at a concentration of 50 μg/mL. Deionized (DI) water was added to the apical chamber of a Transwell® insert and the fluorescence intensity of the polyacrylamide hydrogel layer was monitored, using a fluorescence confocal microscope (Leica, SP8). The fluorescence intensity was quantified and normalized to the value at the initial time point, which corresponds to the time at which monitoring the fluorescent intensity was started. The polyacrylamide hydrogel layer thickness was quantified by measuring the thickness of the fluorescent region.

### Rheological Characterization of the Polyacrylamide Hydrogel.

To characterize the rheological properties of the polyacrylamide hydrogel layer, an affine network model was used. The affine network model assumes that the relative deformation of each individual polymer strand equals the macroscopic deformation of the whole network. The stiffness of the polyacrylamide hydrogel can be expressed with Young's modulus which is calculated using Equation (1), wherein E is the Young's modulus, G is the shear modulus, and v is the Poisson's ratio:

$$E = 2 G ( 1 + v ) \qquad Equation ( 1 )$$

A Poisson's ratio of 0.5 was employed for the calculation of Young's modulus, as Poisson's ratio of rubberlike materials such as hydrogels is usually close to 0.5. Therefore, Young's modulus of the polyacrylamide hydrogel can be estimated as three times the shear modulus. The shear modulus is defined as the ratio of shear stress to shear strain.

A hydrogel disk was prepared for rheological measurements. First, 800 μL of polyacrylamide hydrogel precursor solution was added to a well in a 12-well culture plate and the polyacrylamide hydrogel precursor solution was covered using a circular glass coverslip of 21 mm in diameter. After 30 min, the coverslip was removed, leaving a polyacrylamide hydrogel layer of ˜2 mm in thickness. From the polyacrylamide hydrogel layer, a disk of 8 mm in diameter was cut. The disk was then loaded into a stress-controlled rheometer (Anton Paar, MCR 302) with a plate-plate geometry of 8 mm in diameter, and the polyacrylamide hydrogel peripheral was covered with mineral oil to avoid evaporation. The dynamic mechanical properties were quantified using oscillatory shear measurements at a fixed strain of 0.5% while varying the frequency from 0.1 rad/see to 100 rad/see (frequency sweep from 0.1 rad/see to 100 rad/sec). Within the frequency range explored, the shear modulus was nearly independent of the shear frequency. Therefore, the shear modulus was taken at the lowest shear frequency, 0.1 rad/see, as the equilibrium shear modulus G of the polyacrylamide hydrogel.

### Mesh Size.

The polyacrylamide hydrogel includes long polymer chains that are chemically crosslinked by acrylamide and crosslinking agents to generate a three-dimensional network, as shown in FIG. 8. In addition to polymer networks and crosslinks, polyacrylamide hydrogels have vacancies that allow substances to pass through. Mesh size (8) is the linear distance between two adjacent crosslinks.

The mesh size may be determined using Equation (2), wherein where G is shear modulus, kg is the Boltzmann constant, and T is the absolute temperature.

$$ξ \approx {( \frac{{k}_{B} T}{G} )}^{\frac{1}{3}} \qquad Equation ( 2 )$$

### Cell Culture.

Cells were cultured according to the method described in conjunction with FIG. 11. The polyacrylamide hydrogel layer 120 was first prepared on the support surface 118 of the porous support 110 according to steps 202-208 in FIG. 11. The porous support 110 was a Transwell® insert. The polyacrylamide hydrogel layer 120 was then coated with coating 150 comprising type IV collagen in advance of Human Bronchial Epithelial Cell (HBEC) culture (steps 302 and 304 in FIG. 11). In particular, the outer surface 122 of the polyacrylamide hydrogel layer 120 was activated using sulfosuccinimidyl 6-(4′-azido-2′-nitrophenylamino) (sulfo-SANPAH, Thermo Scientific, Cat. No. 22589) as a photoactivable heterobifunctional crosslinker, which allowed covalent conjugation of the type IV collagen to the outer surface 122 of the polyacrylamide hydrogel layer 120. Specifically, for each gel-coated Transwell® insert, 100 μL of 2 mg/mL sulfo-SANPAH (MilliporeSigma, Cat. No. D2650) solution in DI water was added to the outer surface 122 of the polyacrylamide hydrogel layer 120. The polyacrylamide hydrogel layer 120 was then exposed to UV light (37 mW, 365 nm) for 5 minutes, thereby activating the outer surface 122 of the polyacrylamide hydrogel layer 120. The polyacrylamide hydrogel layer 120 with the activated outer surface was then rinsed with DI water. For each well 104 with an activated polyacrylamide hydrogel outer surface, 150 μL of 0.005 mg/mL type IV collagen (MilliporeSigma, Cat. No. C7521) solution in DI water was added to form coating 150, and the air-liquid-interface culture apparatus 102 was stored at 4° C. overnight to ensure adequate collagen coating. Then, the polyacrylamide hydrogel layer 120 and coating 150 were sterilized with UV light for 15 min.

The cell culture was then prepared according to steps 308-310 of FIG. 11. Primary HBECs were obtained from Marsico Lung Institute Tissue Procurement and Cell Culture Core at the University of North Carolina at Chapel Hill. For all experiments, passage 2 cells were used, as cells beyond passage 2 may lose their ability to differentiate. The cells (HEBCs) were seeded at a density of 4.2×10⁴ cells/insert, and expansion medium (PneumaCult™-Ex Plus Medium, STEMCELL Technologies, Cat. No. 05040) was added to both the upper nutrient compartment and the lower nutrient compartment of the system for culturing cells. After 5-7 days, the HBECs reached over 90% confluence. The cultures were then transitioned to air-liquid-interface by removing the expansion medium from the upper nutrient compartment and replacing the expansion medium in the lower nutrient compartment with air-liquid-interface culture medium (PneumaCult™-ALI Medium, STEMCELL Technologies, Cat. No. 05001). After 2 weeks of air-liquid-interface culture, mucus started to accumulate and the mucus was then washed away three times per week. To wash away the mucus, 500 μL of Dulbecco's phosphate-buffered saline (DPBS, Gibco 14-200-075) was first added to each insert. Then, the cell culture was incubated for 15 min. Finally, the DPBS in the upper nutrient compartment was aspirated. The washing process was repeated a total of three times for each washing.

### Immunofluorescence Microscopy.

Immunofluorescence staining was used to label the cells in a well-differentiated HBEC culture. To prepare for the staining, the culture in each Transwell® was washed three times using DPBS. For each washing, 500 μL of DPBS was added to the apical chamber and the culture was incubated at 37° C. for 20 min. After washing, 1 mL of 4% w/v paraformaldehyde (Thermo Scientific, Cat. No. AA47392-9M) was added to the upper nutrient compartment and the culture was incubated at room temperature for 15 min to fix the cells. Then, 1 mL of 0.2% v/v Triton X-100 solution (Thermo Scientific, Cat. No. AAA16046AP) was added and the culture was incubated at room temperature for 30 min to permeabilize the fixed cells. Next, 1 mL of 3% (v/v) donkey serum (Abcam, Cat. No. AB7475) in DPBS solution was added, and the culture was incubated at room temperature for 1 hour to block non-specific binding of antibodies to cells.

Then, mouse anti-mucin 5AC (MUC5AC) antibody (ThermoFisher, Cat. No. MA5-12178) was used to label mucin granules in goblet cells and rabbit anti-acetylated tubulin (AcTUB) antibody (Invitrogen, Cat. No. MA5-33079) was used to label tubulin in the cilia of ciliated cells. In particular, 200 μL of primary antibody solution containing 10 μg/mL anti-MUC5AC antibody and 40 μg/mL of anti-AcTUB antibody was added to each well. The culture was maintained at 4° C. overnight to ensure the binding of the primary antibody to the antigens. Afterward, the cell culture was rinsed with DPBS three times and 200 μL of secondary antibody solution, which contains 10 μg/ml donkey anti-rabbit secondary antibody conjugated with Alexa Fluor 647 (Abcam, Cat. No. ab150075) and 10 μg/mL donkey anti-mouse secondary antibody conjugated with Alexa Fluor 488 (Abcam, Cat. No. ab150105), was added to each well. The culture was incubated at room temperature for 1 hour to allow the secondary antibodies to bind to the primary antibodies. After the incubation, the culture was rinsed with DPBS three times. After staining with secondary antibodies, 200 μL of 1 μg/mL 4′,6-diamidino-2-phenylindole (DAPI, Invitrogen, Cat. No. D3571) solution in DI water was added to each well to stain the cell nuclei. The culture was again incubated at room temperature for 5 min, and then the culture was washed with DPBS three times.

To prepare the culture for imaging, the Transwell® insert membrane was excised and mounted on a glass cover slide. About 1 mL of FluorSave oil (MilliporeSigma, Cat. No. 345789) was added to completely cover the membrane, and then a coverslip was placed and the edges of the mounting were sealed using wax. A multi-channel fluorescence confocal microscopy (Leica SP8) was used to image the cell culture. During imaging, a sequential scan between frames was used to avoid the overlap of fluorescence from different channels. For each code of cell culture, five regions of interest (ROIs) were randomly selected from five wells with one ROI from each well. Five codes of cells from different healthy donors were used to ensure statistical significance. For each culture, a 10× dry objective was used to image a ROI with the dimension of 1162.5×1162.5 μm²; this allowed for imaging of about 4000 cells/culture and a total number of ˜10⁵ cells for each condition to avoid biased measurement of the cell population. To image the cilia of ciliated cells for a control sample, a 63× oil objective (numerical aperture 1.4) was used to obtain a high-resolution image of individual cilia. The fraction of each cell type was calculated by dividing the cell area by the total area of the image.

### Transepithelial Electric Resistance (TEER) Measurement.

The TEER was measured using an EVOM2 epithelial voltohmmeter (World Precision Instruments, Sarasota, FL). Before each measurement, the electrodes were serialized with 70% ethanol, rinsed using sterilized DI water, and then dried. For each culture, 100 μL of DPBS was added to the upper nutrient compartment of the Transwell® to form an electrical circuit with the liquid in the basal chamber. First, the electric resistance of a blank Transwell® with or without gel coating was measured. Then, to measure the electric resistance across the HBEC culture, the electrodes were placed into the medium on both the upper nutrient compartment and the lower nutrient compartment of the cell culture without disrupting the cell surface. To obtain TEER for HBECs, the measured resistance was subtracted by the value of a blank Transwell® with or without gel and multiplied by the area of the Transwell®. The measurements were performed on air-liquid-interface days 1, 2, 3, 4, 5, 6, 7, 14, 21, and 28 to quantify the dependence of TEER values on culture time.

### Quantification of HBEC Migration and Cell Density.

To monitor the migration of HBECs, a confocal microscope equipped with a 10× dry objective was used to acquire phase contrast images. The phase contrast mode allowed for detecting the differences in the refractive index of cell body and junction, enabling label-free imaging of HBECs at their natural state. Moreover, the confocal microscope was equipped with an environmental chamber with temperature, CO₂, and humidity control to allow for long time imaging. For each condition, HBECs were imaged at 1 frame/min for 60 min on air-liquid-interface days 7, 14, and 21. Particle imaging velocimetry (PIV) to was used to quantify the velocity fields associated with collective cell migration (48). Interrogation windows of 64×64 pixels with 50% overlap were used; this window size allowed for a spatial resolution of ˜ 16 pixels (9 μm).

To measure cell density, for a randomly selected ROI, the number of cells were counted manually. For cells on the four borders of the ROI, only cells on the upper and left borders were included. The cell density was calculated as the total number of cells divided by the area of ROI. For all the measurements, cells from 4-6 healthy donors were used to ensure statistical significance.

### Measurement of the Cell Shape Index.

The cell shape index S was defined as S=P/\sqrt{A}, where P is the cell perimeter and A is the cell area. ImageJ was used to trace the cell contour to measure the cell perimeter and area to calculate the cell shape index. For each condition, 100 cells from 5 wells were randomly chosen representing 5 healthy donors to ensure statistical significance.

### Statistical Analysis.

Statistical analysis was performed using the one-way analysis of variance (ANOVA) method.

### EXAMPLES

The following examples illustrate one or more features of the present disclosure. It should be understood that these examples are not intended to limit the scope of the disclosure or the appended claims in any manner.

### Materials and Methods

Materials: Acrylamide monomer solution (Cat. No. A4058, 40% w/v), N,N′-methylenebisacrylamide solution (Cat. No. M1533, 2% w/v), deionized (DI) water, ammonium persulfate (Cat. No. 248614) N,N,N′,N′-tetramethylethylenediamine (Cat. No. T9281 were purchased from MilliporeSigma. Transwell® inserts (Cat. No. 3401) were purchased from Corning. Fluorescein isothiocyanate-labeled dextran with a molecular weight of 70 kDa (Cat. No. D1823) was purchased from Invitrogen. Sulfo-SANPAH and fluorescent labeled dextran (Texas Red™, MW=70,000 Da) were purchased from Fisher Scientific. Type I collagen solution (PureCol®, 3 mg/ml) was purchased from Advanced Biomatrix. Type IV collagen (Cat. No. C7521) was purchased from MilliporeSigma. PneumaCult™-ALI Medium and PneumaCult™-Ex Plus Basal Medium were purchased from STEMCELL Technologies. Dulbecco's phosphate buffered saline (DPBS, without calcium) was purchased from Fisher Scientific. Paraformaldehyde (purified, Spectrum™), bovine serum albumin (BSA, heat shock treated), 4′,6-Diamidino-2-Phenylindole (DAPI), and dithiothreitol (DTT) were brought from Fisher Scientific. Triton X-100 detergent was purchased from Simga-Aldrich.

Porous Support: Transwell® inserts (12 mm insert, 12 well plate, 0.4 μm polyester membrane, tissue culture treated) were purchased from Corning.

### Example 1: Preparing the Polyacrylamide Hydrogel

To synthesize the polyacrylamide hydrogel with a prescribed stiffness, a polyacrylamide hydrogel precursor solution was prepared by mixing acrylamide solution, N,N′-methylenebisacrylamide solution, and deionized (DI) water (Millipore), according to the volumes listed in Table 1. To initiate polymerization, a final concentration of 0.12% w/v ammonium persulfate was added as a catalyst, and 1.2% v/v N,N,N′,N′-tetramethylethylenediamine was added as an initiator.

TABLE 1
Composition of Reactants for the Synthesis
of Polyacrylamide Hydrogel Samples

| | Total | | | | | |
|---|---|---|---|---|---|---|
| | Monomer | | | | | |
| | Solutions | 40% AAm | 2% BIS | TEMED | 10% APS | H₂O |
| Sample | Vol. % | (μL) | (μL) | (μL) | (μL) | (μL) |
| 1A | 7.5 | 75 | 75 | 12 | 12 | 826 |
| 1B | 10.0 | 100 | 100 | 12 | 12 | 776 |
| 1C | 12.5 | 125 | 125 | 12 | 12 | 726 |
| 1D | 15.0 | 150 | 150 | 12 | 12 | 676 |
| 1E | 17.5 | 175 | 175 | 12 | 12 | 626 |
| 1F | 20.0 | 200 | 200 | 12 | 12 | 576 |
| 1G | 22.5 | 225 | 225 | 12 | 12 | 526 |

Transwell® inserts were used as porous supports, and the polyacrylamide hydrogel layers were prepared according to the method described in relation to FIG. 7. To coat a Transwell® insert with a polyacrylamide hydrogel layer of 100 μm thickness, 30 μL of the polyacrylamide hydrogel precursor solution was applied to the center of the Transwell® as quickly as possible to minimize the polymerization of the polyacrylamide hydrogel precursor solution before it was applied to the Transwell® insert. In parallel, a circular coverslip of 11 mm in diameter was prepared by punching three layers of Scotch® tape and using the punched three layers of Scotch® tape as a coverslip to spread the reaction mixture. Because the coverslip is hydrophobic and has a diameter slightly smaller than that of the Transwell® insert (which is 12 mm), this process flattens the gel surface, and prevents the reaction mixture from being exposed to oxygen, which impairs free radical polymerization. After 30 min, the coverslip was removed by a hooked syringe, leaving a thin layer of polyacrylamide hydrogel with the prescribed thickness. Afterward, the gel was rinsed using sterilized DI water. 12-well plates containing gel-coated Transwell® inserts were sealed with parafilm and stored at 4° C. for future usage.

### Example 2: Rheological Properties of the Polyacrylamide Hydrogels

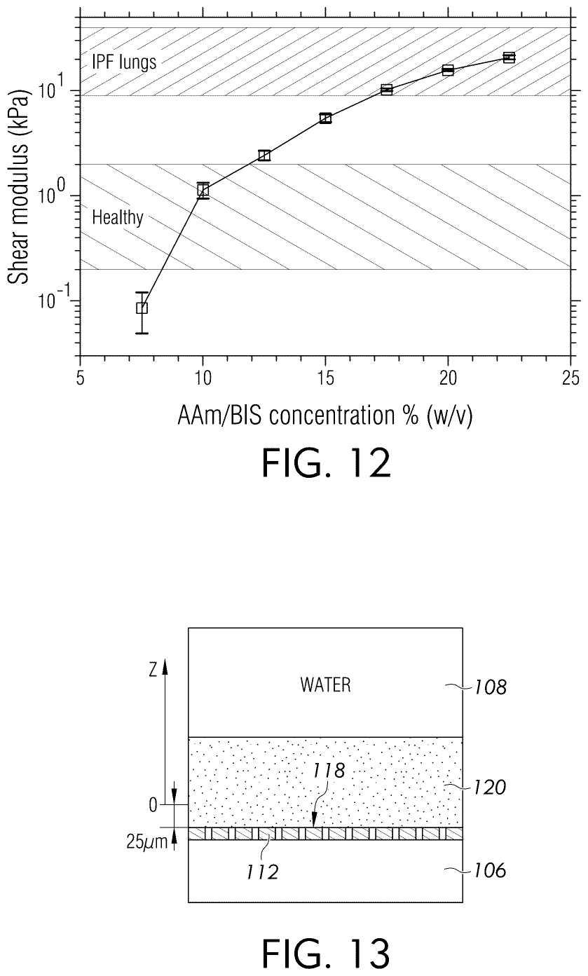

In Example 2, the rheological properties of the polyacrylamide hydrogels of Example 1 were evaluated according to the methods disclosed herein. Measurements for each group were performed three times and the results were averaged. The shear modulus and estimated Young's modulus of the polyacrylamide hydrogels of Example 1 are provided in Table 2. As shown in Table 2, increasing the amount of the monomers (e.g., total concentration of the acrylamide solution and N,N′-methylenebisacrylamide solution) in the hydrogel precursor solution increases the shear modulus and corresponding Young's modulus of the resulting polyacrylamide hydrogel layer. The relationship between shear modulus and AAm/BIS concentration for a polyacrylamide hydrogel layer is graphically depicted in FIG. 12.

TABLE 2
Relationship Between Monomer Concentration in Hydrogel Precursor
Solution and Shear Modulus of the Polyacrylamide Hydrogel

| | Monomer | | |
|---|---|---|---|
| | Solutions | | Estimated |
| | vol. % in | Shear | Young's |
| Sample | precursor | modulus (Pa) | modulus (Pa) |
| 1A | 7.5 | 84.82 | 254.46 |
| 1B | 10.0 | 1139.55 | 3418.65 |
| 1C | 12.5 | 2421.33 | 7264 |
| 1D | 15.0 | 5500.7 | 16502.1 |
| 1E | 17.5 | 10203.83 | 30611.5 |
| 1F | 20.0 | 15705.33 | 47116 |
| 1G | 22.5 | 20842.33 | 62527 |

### Example 3: Permeability of the Polyacrylamide Hydrogel Layers

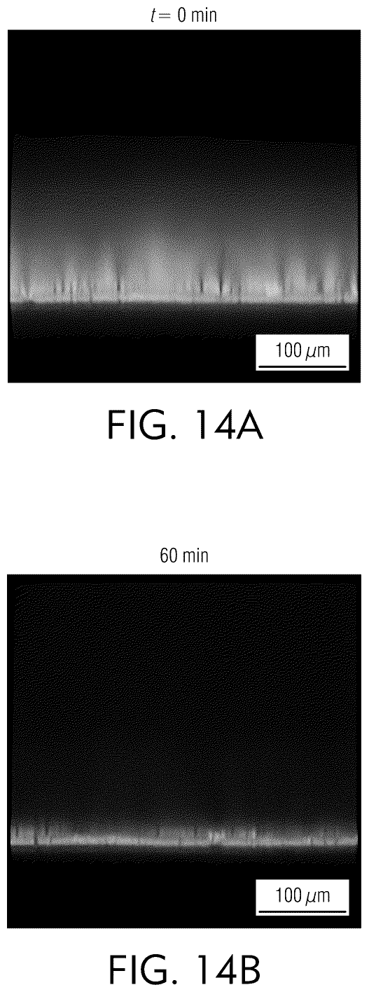

In Example 3, the relationship between the polyacrylamide hydrogel layer thickness and the permeability was determined. Referring to FIG. 13, polyacrylamide hydrogel layers 120 including 20 vol. % of the monomer solutions (i.e., combination of the acrylamide monomer solution and the methylenebisacrylamide solution) were prepared, with the thickness of the polyacrylamide hydrogel layers 120 being 100 μm, 250 μm, 500 μm, and 1000 μm. Fluorescein isothiocyanate-labeled dextran (Texas Red) with a molecular weight of 70 kDa, which is a branched polymer with a hydrodynamic diameter of about 6.4 nm, which is close to the diameter of albumin, was added to the polyacrylamide hydrogel precursor solution prior to formation of the polyacrylamide hydrogel layers 120. Because dextran molecules are prone to concentrate on the surface 118 of the plastic membrane of the porous polymer plate 112 due to unspecific interactions, the fluorescence intensity was measured for regions at positions greater than about 25 μm above the membrane to avoid the effects of concentrated dextran on the measurements. FIG. 14A shows an optical image of the fluorescently labeled polyacrylamide hydrogel layer at a start time, and FIG. 14B shows an optical image of the fluorescently labeled polyacrylamide hydrogel layer one hour after the start time.

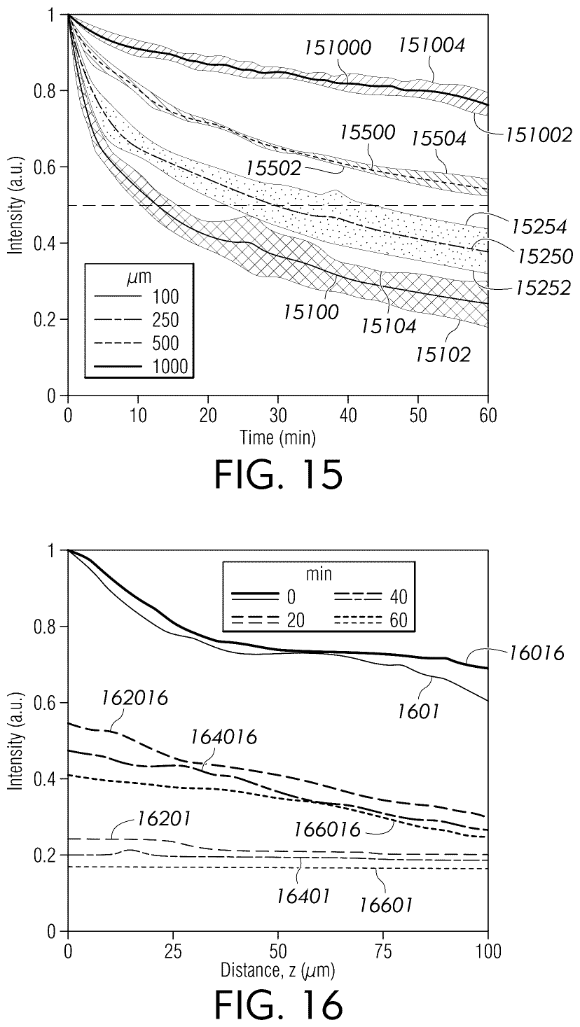

To describe the transport properties of the polyacrylamide hydrogel layer, the half-decay time t, at which the fluorescence intensity of the polyacrylamide hydrogel decreases to 50% of the initial value, was used. For polyacrylamide hydrogel layers having a stiffness of 1 kPa, as the thickness increased from 100 μm to 250 μm, t increased by nearly three times from 10 to 30 min, as shown in FIG. 15. In FIG. 15, line 151000 represents the polyacrylamide hydrogel layer with a thickness of 1000 μm, and 151004 and 151002 outline the upper and lower error limits, respectively, line 15500 represents the polyacrylamide hydrogel layer with a thickness of 500 μm, and 15504 and 15502 outline the upper and lower error limits, line 15250 represents the polyacrylamide hydrogel layer with a thickness of 250 μm, and 15254 and 15252 outline the upper and lower error limits, and line 15100 represents the polyacrylamide hydrogel layer with a thickness of 100 μm, and 15104 and 15102 outline the upper and lower error limits. Further increasing the polyacrylamide hydrogel layer thickness to 500 μm dramatically increased the value τ to nearly 60 min (FIG. 15); this time is much longer than the typical nutrient supply rate required for optimal cell culture. At a polyacrylamide hydrogel layer thickness of 1000 μm, the intensity decreased to 75% of its initial value after one hour (FIG. 15). These results confirm the understanding that thinner polyacrylamide hydrogel layers permit more efficient mass transport of nutrients through the polyacrylamide hydrogel layer.

Moreover, even for the polyacrylamide hydrogel layer having a stiffness of 16 kPa, 100 μm thickness allowed for relatively efficient mass transport with τ≈20 min, as evidenced by the fluorescence intensity profiles across the polyacrylamide hydrogel layer at different time scales, as shown in FIG. 16. In FIG. 16, line 1601 represents the polyacrylamide hydrogel layer with a stiffness of 1 kPa after 0 minutes, line 16201 represents the polyacrylamide hydrogel layer with a stiffness of 1 kPa after 20 minutes, line 16401 represents the polyacrylamide hydrogel layer with a stiffness of 1 kPa after 40 minutes, line 16601 represents the polyacrylamide hydrogel layer with a stiffness of 1 kPa after 60 minutes, line 16016 represents the polyacrylamide hydrogel layer with a stiffness of 16 kPa after 0 minutes, line 162016 represents the polyacrylamide hydrogel layer with a stiffness of 16 kPa after 20 minutes, line 164016 represents the polyacrylamide hydrogel layer with a stiffness of 16 kPa after 40 minutes, and line 164016 represents the polyacrylamide hydrogel layer with a stiffness of 16 kPa after 60 minutes. The determined optimum polyacrylamide hydrogel thickness of ˜100 μm for efficient mass transport is much smaller than 750 μm. These results are consistent with the existing understanding that in native tissues, mammalian cells are typically located within 100-200 μm of blood vessels and that in engineered tissues only cells up to a distance of 200 μm away from the media have access to sufficient nutrients.

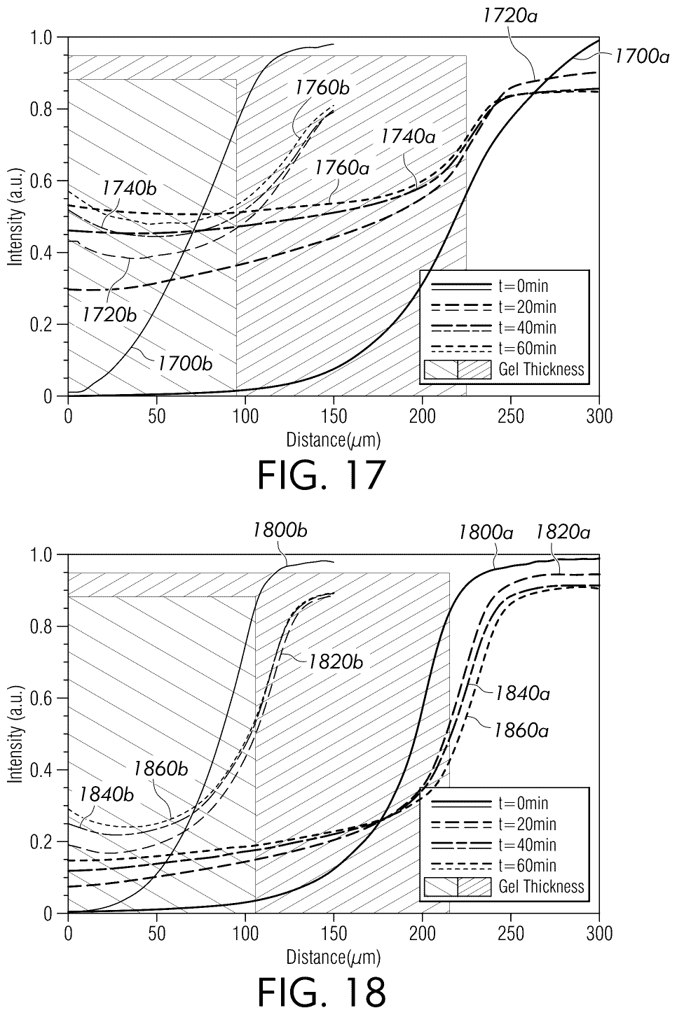

The impact of polyacrylamide hydrogel layer stiffness on diffusion was also investigated. Normalized diffusion profiles were computed and are shown in FIG. 17 for a polyacrylamide hydrogel layer made with 10 vol. % monomer (AAm/BIS 10% hydrogel) and FIG. 18 for a polyacrylamide hydrogel layer made with 20% monomer (AAm/BIS 20% hydrogel).

In FIG. 17, line 1700b represents a 100 μm thick polyacrylamide hydrogel layer after 0 minutes, line 1720b represents a 100 μm thick polyacrylamide hydrogel layer after 20 minutes, line 1740b represents a 100 μm thick polyacrylamide hydrogel layer after 40 minutes, line 1760b represents a 100 μm thick polyacrylamide hydrogel layer after 60 minutes, line 1700a represents a 250 μm thick polyacrylamide hydrogel layer after 0 minutes, line 1720a represents a 250 μm thick polyacrylamide hydrogel layer after 20 minutes, line 1740a represents a 250 μm thick polyacrylamide hydrogel layer after 40 minutes, and line 1760a represents a 250 μm thick polyacrylamide hydrogel layer after 60 minutes.

In FIG. 18, line 1800b represents a 100 μm thick polyacrylamide hydrogel layer after 0 minutes, line 1820b represents a 100 μm thick polyacrylamide hydrogel layer after 20 minutes, line 1840b represents a 100 μm thick polyacrylamide hydrogel layer after 40 minutes, line 1860b represents a 100 μm thick polyacrylamide hydrogel layer after 60 minutes, line 1800a represents a 250 μm thick polyacrylamide hydrogel layer after 0 minutes, line 1820a represents a 250 μm thick polyacrylamide hydrogel layer after 20 minutes, line 1840a represents a 250 μm thick polyacrylamide hydrogel layer after 40 minutes, and line 1860a represents a 250 μm thick polyacrylamide hydrogel layer after 60 minutes.

Comparing the intensity change in the 100 μm thick AAm/BIS 10% polyacrylamide hydrogel at x=0 to the total intensity change over one hour suggests that most of the dextran particles diffused rapidly in the first 20 minutes, while it took about 40 minutes for most of the dextran to diffuse through the 250 μm thick AAm/BIS 10% polyacrylamide hydrogel (see FIG. 17). As for the stiffer AAm/BIS 20% polyacrylamide hydrogel, the diffusion rate of dextran was lower (see FIG. 18) compared to the diffusion rates for the AAm/BIS 10% polyacrylamide hydrogel (FIG. 17). Moreover, the difference in the intensity change in the first 20 minutes for the AAm/BIS 20% polyacrylamide hydrogel (FIG. 18) was significantly less than the difference in the intensity change for the AAm/BIS 10% polyacrylamide hydrogel (FIG. 17). Without being bound by theory, it is believed that this is because the AAm/BIS 20% polyacrylamide hydrogel has a smaller mesh size, and so the dextran particles are more easily trapped in the hydrogel network. Yet, the intensity continuously increased during the first hour for the AAM/BIS 20% polyacrylamide hydrogel. Collectively, these results demonstrate that large metabolic biomolecules in nutrient media can diffuse through the polyacrylamide hydrogel layer and reach the cells on the polyacrylamide hydrogel surface.

### Example 4: Permeability vs. Stiffness of the Polyacrylamide Hydrogel

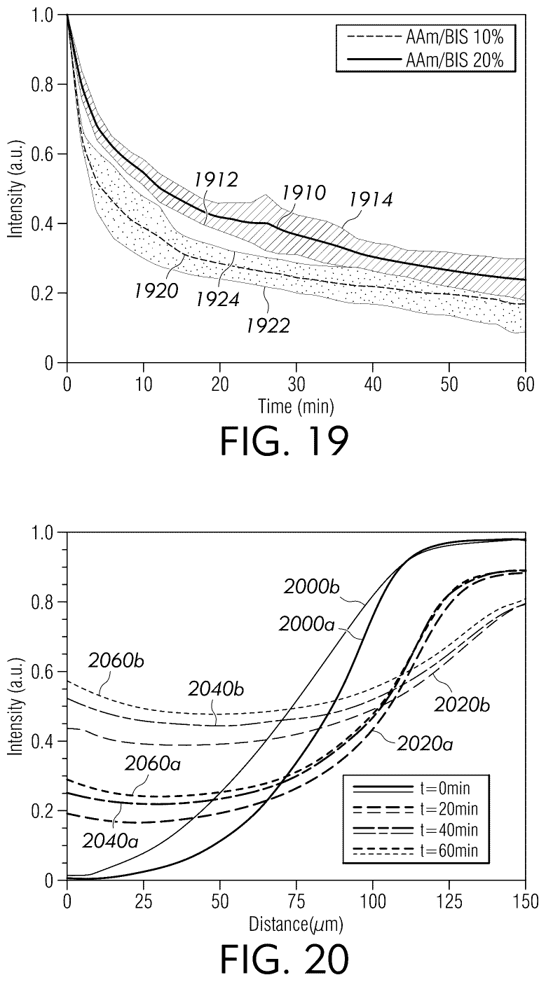

In Example 4, the relationship between stiffness and diffusion properties for the polyacrylamide hydrogel layers was investigated. To determine the relationship between hydrogel stiffness and its diffusion properties, 100 μm thick polyacrylamide hydrogel layers were synthesized with different stiffness (i.e., AAm/BIS 10% and 20% polyacrylamide hydrogels) and the fluorescence intensity was quantified for one-hour diffusion, as shown in FIG. 19. In FIG. 19, line 1910 represents the results for a polyacrylamide hydrogel layer with AAm/BIS 10%, and 1914 and 1912 outline the upper and lower error limits, and line 1920 represents the results for a polyacrylamide hydrogel layer with AAm/BIS 20%, and 1924 and 1922 outline the upper and lower error limits. As shown in FIG. 19, it took longer for dextran to diffuse through the hydrogel layer as the acrylamide and bis-acrylamide concentration increased from 10 vol. % to 20 vol. %. Quantitatively, the half-decay time of fluorescent intensity increased from 5 minutes for the AAm/BIS 10% hydrogel to 12 minutes for the AAm/BIS 20% hydrogel. These results provide evidence that the polyacrylamide hydrogel stiffness is determined by the mesh size of the hydrogel network and thus results in different dextran diffusion rates. It is noted that the standard deviation errors bars of the AAm/BIS 10% hydrogel and the AAm/BIS 20% hydrogel mostly overlap after the first 30 minutes. Without being bound by theory, this is believed to be because fluorescent particles in thinner polyacrylamide hydrogel layers can more easily escape the hydrogel network, resulting in a reduction of the diffusion difference between the hydrogels of different stiffness.

### Example 5: Permeability of Polyacrylamide Hydrogel Layer Applied to the Porous Support

To determine the permeability of polyacrylamide hydrogel layers on the porous support, fluorescent-labeled dextran (Texas Red) was added to water in the lower nutrient compartment 106, as shown in FIG. 10. The diffusion profile of the dextran was quantified by measuring 25 μm above the boundary of the support surface 118 and the polyacrylamide hydrogel layer 120. The intensity was normalized to the maximum intensity of the diffusion curve. The results are shown in FIG. 20. In FIG. 20, line 2000b represents the results for a polyacrylamide hydrogel layer with AAm/BIS 10% after 0 minutes, 2020b represents the results for a polyacrylamide hydrogel layer with AAm/BIS 10% after 20 minutes, 2040b represents the results for a polyacrylamide hydrogel layer with AAm/BIS 10% after 40 minutes, 2060b represents the results for a polyacrylamide hydrogel layer with AAm/BIS 10% after 60 minutes, 2000a represents the results for a polyacrylamide hydrogel layer with AAm/BIS 20% after 0 minutes, 2020a represents the results for a polyacrylamide hydrogel layer with AAm/BIS 20% after 20 minutes, 2040a represents the results for a polyacrylamide hydrogel layer with AAm/BIS 20% after 40 minutes, and 2060a represents the results for a polyacrylamide hydrogel layer with AAm/BIS 20% after 60 minutes.

The results in FIG. 20 show that most of the dextran molecules can diffuse from the water in the upper nutrient chamber 108 (apical chamber) to the polyacrylamide hydrogel layer in the first 20 minutes and the fluorescent intensity gradually reaches equilibrium after 40 minutes. Moreover, the dextran molecules diffuse faster in the AAm/BIS 10% hydrogel compared to AAm/BIS 20% hydrogel, which has greater stiffness.

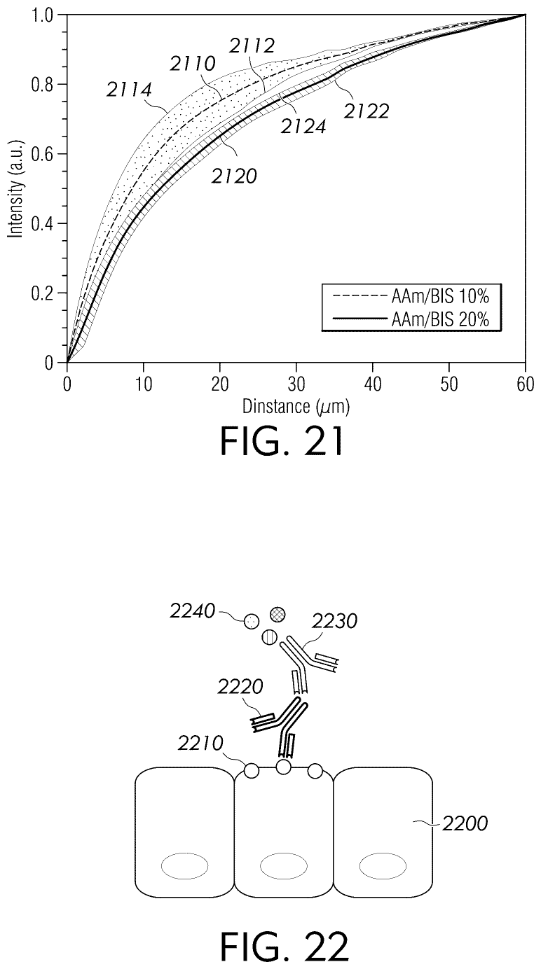

The fluorescent intensity of the hydrogel area was further quantified from z=0 to z=20 μm, which is the farthest place from the dextran particles in the polyacrylamide hydrogel layer, as shown in FIG. 21. In FIG. 21, line 2110 represents the results for a polyacrylamide hydrogel layer with AAm/BIS 10%, and 2114 and 2112 outline the upper and lower error limits, and line 2120 represents the results for a polyacrylamide hydrogel layer with AAm/BIS 20%, and 2124 and 2122 outline the upper and lower error limits. The intensity exhibits a sharp increase to 50% of its maximum intensity at 6.5 and 9 minutes for the AAm/BIS 10% hydrogel and the AAm/BIS 20% hydrogel, respectively. These results validate that the diffusion of large particles in the polyacrylamide hydrogel layer is determined by the gel stiffness, meaning the diffusion properties of the polyacrylamide hydrogel layers remain similar when applying the polyacrylamide hydrogel layers in the air-liquid-interface cell culture system.

### Example 6: Human Bronchial Epithelial Cell Culture: Cell Differentiation

In Example 6, differentiation of human bronchial epithelial cell cultures grown using the air-liquid-interface cell culture systems of the present disclosure were studied. After seeding, Human Bronchial Epithelial (HBE) cells were grown using the following protocol. Firstly, HBE cells were grown in 500 μL PheumaCult-Ex plus basal medium supplemented with 0.1% heparin in an incubator at 37° C. and 5% CO₂. 1 ml basal media was added to the basal chamber of the lower nutrient compartment. The basal medium was changed every other day until cells were 80˜90% confluent. Once the HBE cells in submerged culture reached confluence, the apparatus was lifted to create an air-liquid interface. PheumaCult-Ex plus basal medium was removed from both the upper nutrient compartment and the lower nutrient compartment. Then, 1 mL PneumaCult ALI media was added to the lower nutrient compartment, and the upper nutrient compartment was kept dry.

To visualize the differentiation of airway epithelial cells in a gel-ALI culture system, immunofluorescence staining was conducted to observe the HBE cells on air-liquid-interface Transwell porous supports without polyacrylamide hydrogel layers, on the air-liquid-interface at the upper surface of a polyacrylamide hydrogel layer formed with 10 vol % monomer solution (PAAm AAm/BIS 10% hydrogel), and on the air-liquid-interface of the PAAm AAm/BIS 20% hydrogel after four weeks of ALI culture. Immunostaining is a standard method to detect antigens 2210 (proteins) in cells 2200 based on the specific binding of a primary antibody 2220 to an antigen 2210, as shown in FIG. 22. This process involves using a primary antibody 2220 that targets a specific antigen 2210 on cells 2200. Then a second antibody 2230 is applied to bind to the primary antibody 2200 as well as to help amplify the detection signal. The secondary antibody 2230 is also conjugated with the fluorescent markers 2240 and can be visualized using a fluorescence microscope.

Immunostaining was conducted by first immersing the cells in paraformaldehyde (PFA, 4%) for 15 mins to fix the cells, and then adding dithiothreitol (DTT, 10 mM) to treat the cells for 15 mins. Afterward, the cells were immersed in permeabilizing solution (0.2% Triton X-100 in PBS). Then, the cells were blocked using 3% BSA for one hour to prevent non-specific staining of antibodies. The BSA was then removed and the cells were incubated with rat anti-alpha tubulin primary antibody 1:250 dilution in 3% BSA and mouse anti mucin 5AC (MUC5AC) primary antibody 1:1000 dilution in 3% BSA overnight at 4° C. Anti-acetylated tubulin antibodies were applied to label ciliated cells and anti-MUC5AC antibodies were used to mark goblet cells. After washing the cells with PBS for 7 mins three times, the cells were stained with secondary antibody solution in 3% BSA for one hour. Finally, the cells were incubated with 4′,6-diamidino-2-phenylindole (DAPI) 1:1000 solution in sterilized DI water for 5 mins to stain the nuclei. The cells were visualized using confocal microscope.

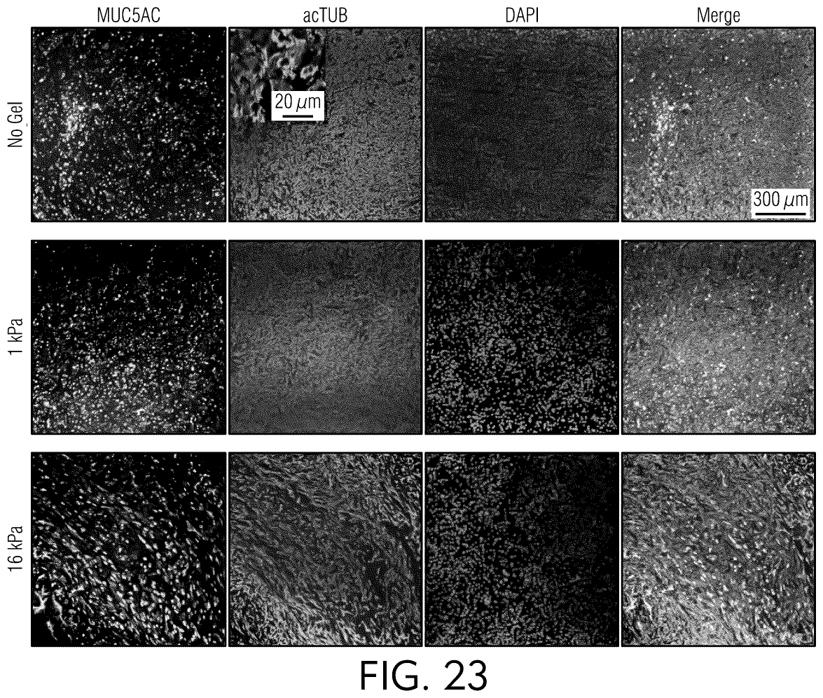

As shown in the fluorescent microscopy images of FIG. 23, basal cells growing on the transwells in the classical ALI culture (i.e., without the polyacrylamide hydrogel layer) were fully differentiated into a pseudostratified epithelium with ciliated cells and goblet cells. The goblet cells are shown in the left most column of FIG. 23, and were stained using MUC5AC. The ciliated cells are shown in the second column from the left of FIG. 23, and were stained using acTUB as a tublin marker. Cell nuclei are shown in the second column from the left of FIG. 23, and were stained using DAPI. A merge of the three stains is shown in the right most column of FIG. 23. The cell survival indicated that the transport of the nutrients in the cell culture media are sufficient to pass through the PAAm hydrogel layer and reach the cells. Although HBE cells in the gel-ALI culture system differentiated into goblet cells and ciliated cells, the amount of goblet cells was lower when culturing on polyacrylamide hydrogel with 20% AAm/BIS, compared to the cells culturing on plastic membrane. This suggests that substrate stiffness might affect the cell behavior.

Based on these observations, the polyacrylamide hydrogel culture system is demonstrated to be cyto-compatible for HBECs growth and differentiation. To further investigate the impact of ECM-mimicking substrate stiffness on HBE cells, HBE cells were observed under different gel stiffnesses by quantifying cell density, cell migration and cell shape index.

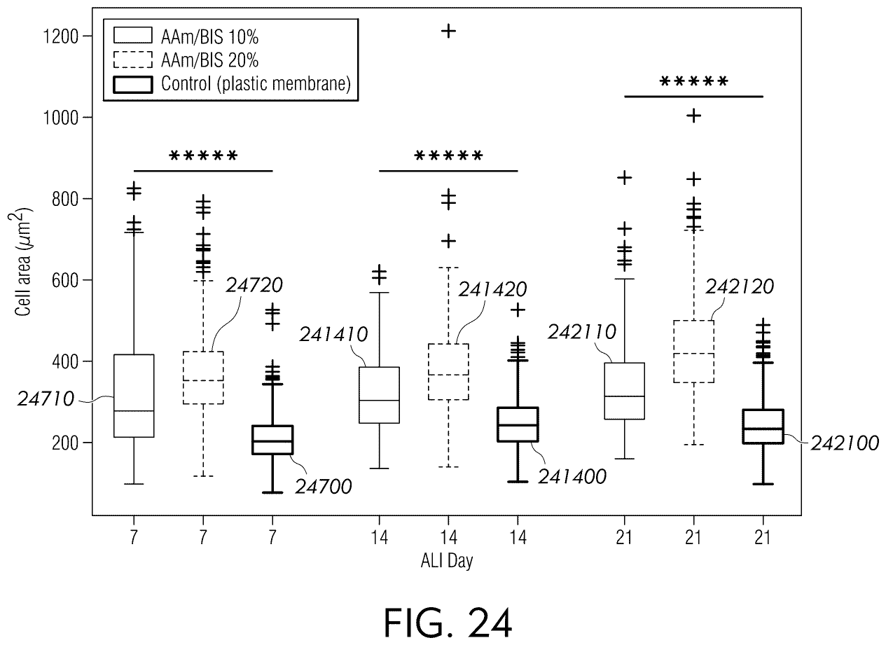

To examine the morphological changes of HBE cells under different substrate stiffness, the cell area on ALI days 7, 14, and 21 was quantified, as shown in FIG. 24. In FIG. 24, 24710 represents the results for a polyacrylamide hydrogel layer with AAm/BIS 10% for ALI day 7, 24720 represents the results for a polyacrylamide hydrogel layer with AAm/BIS 20% for ALI day 7, 24700 represents the results for no polyacrylamide hydrogel layer for ALI day 7, 241410 represents the results for a polyacrylamide hydrogel layer with AAm/BIS 10% for ALI day 14, 241420 represents the results for a polyacrylamide hydrogel layer with AAm/BIS 20% for ALI day 14, 241400 represents the results for no polyacrylamide hydrogel layer for ALI day 14, 242110 represents the results for a polyacrylamide hydrogel layer with AAm/BIS 10% for ALI day 21, 242120 represents the results for a polyacrylamide hydrogel layer with AAm/BIS 20% for ALI day 21, and 242100 represents the results for no polyacrylamide hydrogel layer for ALI day 21. Quantitatively, cells grown on PAAm hydrogel had a larger cell area (400 μm²/cell on AAm/BIS 20% gel) compared to cells grown directly on a plastic membrane on ALI day 21 (210 μm²/cell). For cells grown on the gel-ALI system, cells on AAm/BIS 10% hydrogel had slightly larger cell area, with a median value of 430 μm²/cell. These results indicate that substrate stiffness is associated with cell area and cell growth. Yet, the increased cell area on soft substrate slows down cell proliferation. Nevertheless, the cells on PAAm hydrogel have the capacity to differentiate into ciliated cells and goblet cells.

### Example 6: HBEC Culture: Cell Density

Cell density may be used as an indicator for cell responses from different biophysical cues. An abnormal increase or decrease of cell density can reflect the inability of the cells to accommodate cell proliferation or differentiation. Average cell size can also be used to observe cell behavior since increased cell density is associated with the decrease of average cell size.

To study the response of HBE cells to substrate stiffness, HBE cells were cultured on plastic membrane, AAm/BIS 10% PAAm hydrogel, which is equivalent to the stiffness of lung tissue, and AAm/BIS 20% PAAm hydrogel that mimics the stiffness of IPF lungs. Bright field images of the HBE cells were obtained using phase-contrast mode on ALI days 7, 14 and 21. Four images were randomly taken from each well for three separate wells. Cell density was measured by choosing three random areas of the cell images and then quantifying the number of cells in those areas.

The results show that HBE cells cultured directly on the plastic membrane have higher cell density compared to cells grown on soft PAAm hydrogel layer after two weeks of ALI culture, as shown in FIG. 23. Moreover, the density of HBE cells cultured on AAm/BIS 20% gel was greater than the density of cells on AAm/BIS 10% gel. This suggests that lower substrate stiffness slows down cell proliferation. Likewise, the quantification of average cell area also demonstrated this result.

Despite the efficient transport of protein mimics, the ultimate test of the gel-ALI system is to examine whether HBECs can grow and differentiate into a pseudostratified bronchial epithelium. To this end, the surface of the polyacrylamide hydrogel was activated using sulfosuccinimidyl 6-(4′-azido-2′-nitrophenylamino) hexanoate (sulfo-SANPAH), such that the polyacrylamide hydrogel surface was conjugated with type IV collagen, a standard ECM protein for culturing primary HBECs. Similar to the classical ALI culture, primary HBECs were plated and cultured by adding medium to both the apical and basal sides for about one week to reach >90% confluence. Afterward, HBECs were transitioned to ALI by removing the apical medium and maintaining medium on the basal side only. The cells were cultured for 4 weeks after which HBECs were expected to fully differentiate to a pseudostratified epithelium.

Immunostaining was performed to identify the population of cells cultured on substrates of various stiffnesses. As predicted, for the control group without a polyacrylamide hydrogel coating, the HBECs fully differentiate into a pseudostratified epithelium consisting of ciliated cells and mucus-secreting goblet cells, as shown by the fluorescence images for each cell type, as shown in the top row of FIG. 23. Quantitatively, for the control group the fraction of ciliated cells was 0.29±0.17, whereas the fraction of goblet cells was 0.13±0.08. These values are consistent with literature data that for in vitro HBEC culture the fractions of ciliated and goblet cells, respectively, are about 0.30 and 0.10-0.15.

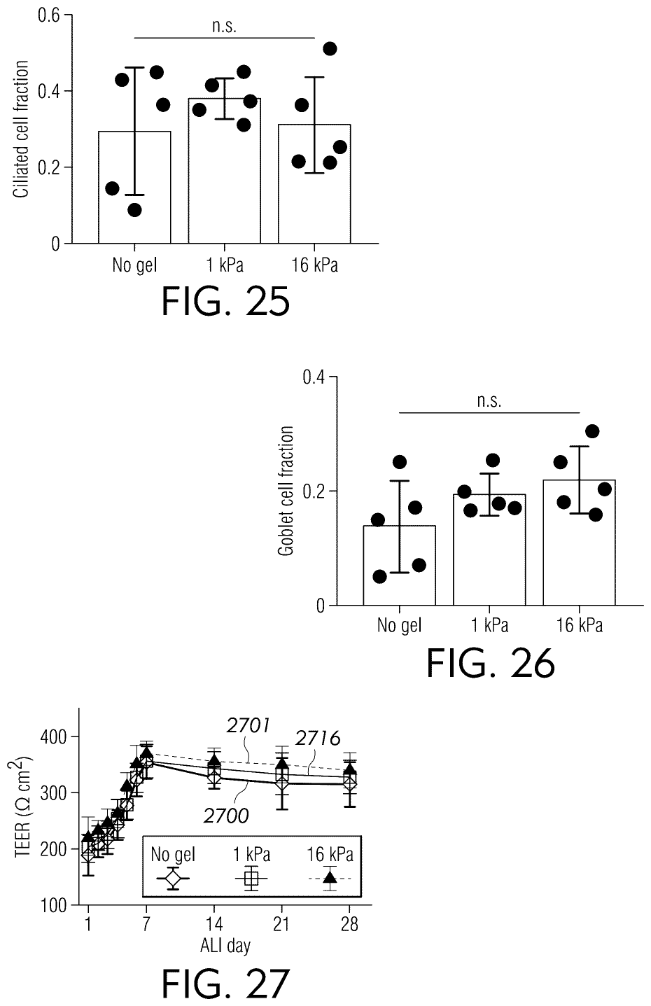

Similarly, HBECs differentiated into pseudostratified epithelia on polyacrylamide hydrogels of 1 kPa and 16 kPa, as shown in the middle and the bottom rows of FIG. 23. Interestingly, regardless of the polyacrylamide hydrogel coating, the fraction of ciliated cells was in the range of 0.3-0.4, as shown in FIG. 25. Moreover, the fraction of goblet cells was around 0.2 and there was no significant difference among substrates of various stiffnesses, as shown in FIG. 26. The stained ciliated and goblet cells together accounted for about 50% of the total cell population; the remain cells were other cells types such as basal cells and club cells.

In parallel, the transepithelial electrical resistance (TEER) of HBEC cultures was quantified during the cells' various stages of growth and differentiation. The contribution from the polyacrylamide hydrogel layer was subtracted to obtain the TEER values for the HBECs. Up to ALI Day 7 the HBEC TEER value increased nearly linearly from ˜200 to 350 (2 cm2, reflecting the proliferation of HBECs (FIG. 27). In FIG. 27, 2700 represents a sample with no polyacrylamide hydrogel, 2701 represents a sample with a stiffness of 1 kPa, and 2716 represents a sample with a stiffness of 16 kPa. After ALI Day 7, the TEER value slightly decreased and became stable beyond ALI Day 21, reflecting the complete differentiation of the HBECs. Taken together, these results show that the air-liquid-interface apparatus described herein allows HBECs to differentiate into a pseudostratified epithelium.

### Example 7: HBEC Culture Cell Migration

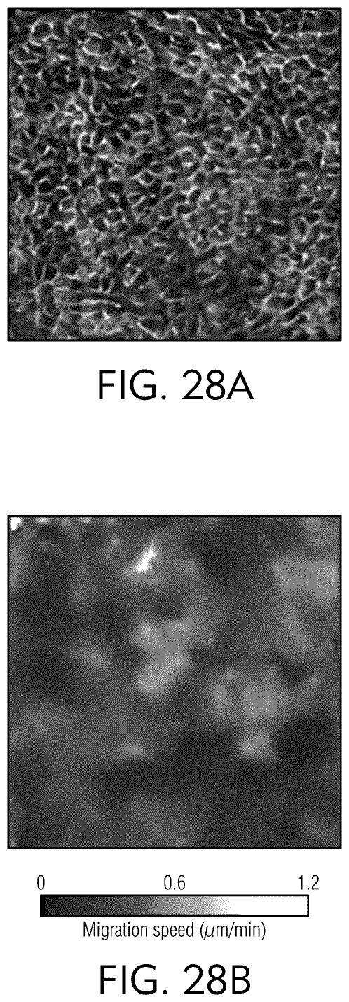

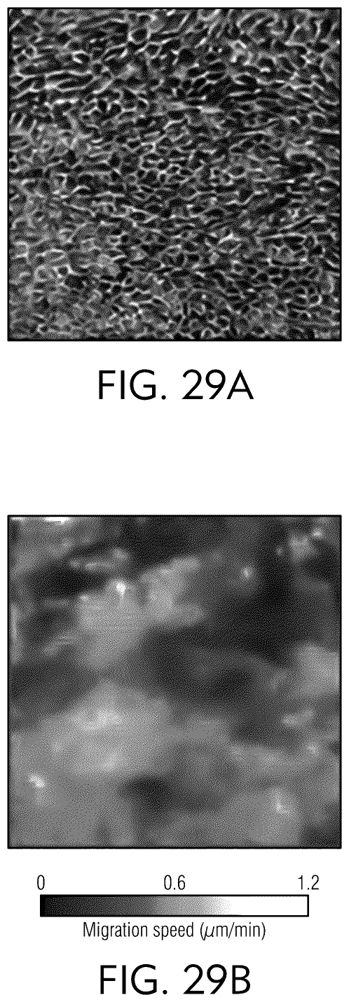

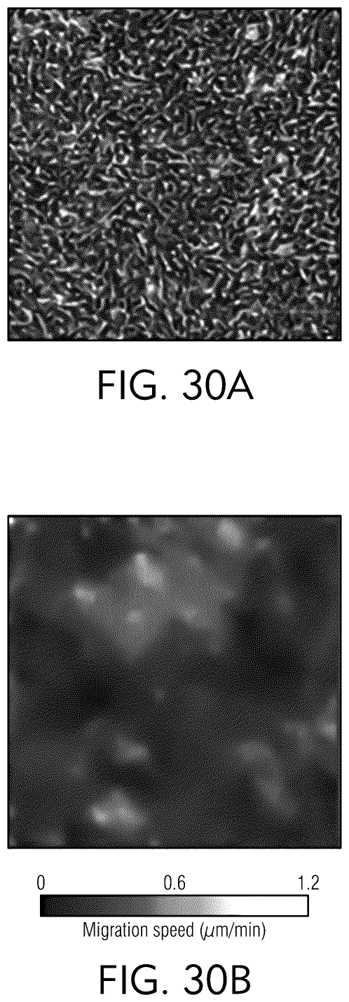

Cell migration is a common process of cell development and maintenance. Cells often migrate in a specific direction when an area needs to differentiate cells to heal a wound or to induce an immune response. Moreover, cell migration may differ with external signals, such as matrix stiffness. HBE cell migration was monitored with a confocal microscope equipped with a cell culture incubator (37° C. and 5% CO₂). Cell migration was monitored under different substrate stiffnesses on ALI days 7, 14 and 21. Time-lapse cell images were taken every 2 minutes for a total of 100 frames. The cell migration speed and migration speed maps were obtained using particle image velocimetry (PIVlab) software. The migration speed maps, shown in FIG. 28B, FIG. 29B, and FIG. 30B, show that there was more cell migration on AAm/BIS 20% polyacrylamide hydrogel than on AAm/BIS 10% polyacrylamide hydrogel or no polyacrylamide hydrogel layer after three weeks of ALI culture.

Cell migration speed was quantified, and HBE cells on AAM/BIS 10% polyacrylamide hydrogel were more migratory compared to cells cultured without a polyacrylamide hydrogel layer on ALI day 21. Additionally, cells on AAm/BIS 20% polyacrylamide hydrogel migrated faster than cells on AAm/BIS 10% polyacrylamide hydrogel. On ALI day 14, HBE cells on AAm/BIS 20% polyacrylamide hydrogel migrated faster than those on AAm/BIS 10% polyacrylamide hydrogel, while there was no significant difference between cells on AAm/BIS 10% polyacrylamide hydrogel and cells that were directly on the porous support. On ALI day 7, cell migration showed no differences among three groups.

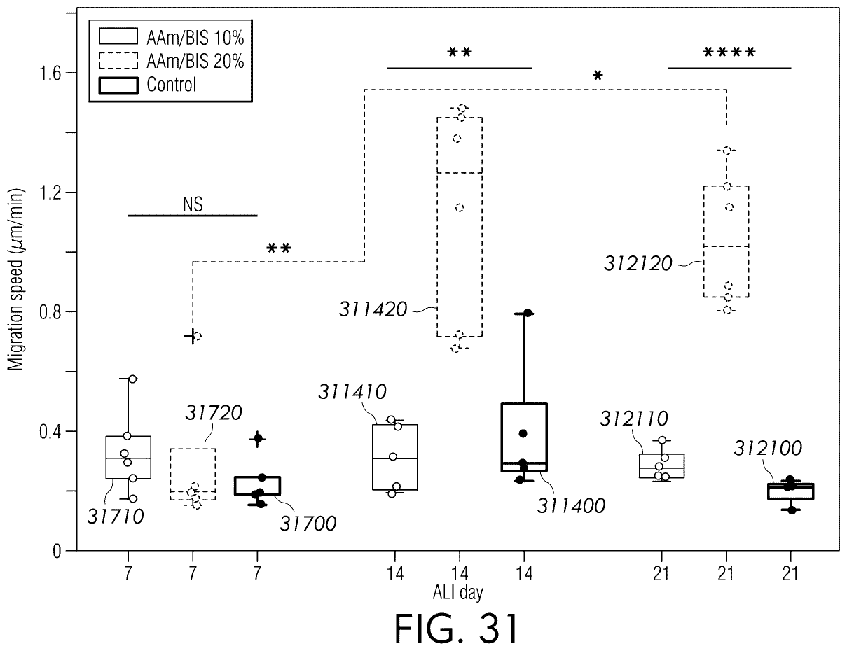

Furthermore, the cell migration speed for all groups increased on ALI day 14, as shown in FIG. 31. In FIG. 31, 31710 represents the results for a polyacrylamide hydrogel layer with AAm/BIS 10% for ALI day 7, 31720 represents the results for a polyacrylamide hydrogel layer with AAm/BIS 20% for ALI day 7, 31700 represents the results for no polyacrylamide hydrogel layer for ALI day 7, 311410 represents the results for a polyacrylamide hydrogel layer with AAm/BIS 10% for ALI day 14, 311420 represents the results for a polyacrylamide hydrogel layer with AAm/BIS 20% for ALI day 14, 311400 represents the results for no polyacrylamide hydrogel layer for ALI day 14, 312110 represents the results for a polyacrylamide hydrogel layer with AAm/BIS 10% for ALI day 21, 312120 represents the results for a polyacrylamide hydrogel layer with AAm/BIS 20% for ALI day 21, and 312100 represents the results for no polyacrylamide hydrogel layer for ALI day 21. Although the increased migration speeds might be attributed to slower proliferation or weaker cell adhesion, the migration speed decreased after three weeks of ALI culture. These results raise the question that whether the substrate stiffness affects cell mobility. Therefore, the cell migration was further studied through the use of cell shape index.

### Example 8: HBEC Culture Cell Shape

The difference in cell migration can be further interpreted with an unjammed-to-jammed transition model. When the cells are in an unjammed state, the cells can move through the tissues and have high mobility. However, when the cells are packed tightly, the shape may transition from an unjammed state to a jammed state. The unjammed-to-jammed transition can be determined by quantifying an index called the cell shape index. The cell shape index, defined as cell perimeter divided by square root of cell area, correlates to cell shape and cell structural stability. High cell shape index occurs when the cells are immature and unstable due to irregular, elongated cell shape. When cell shape index decreases and approaches 3.81, the transition occurs and the cells become static and jammed.

To measure the cell shape index, bright field images were obtained using phase-contrast mode on ALI days 7, 14, and 21. Four images per well from three different wells were randomly chosen. Twenty-five cells in each image were randomly selected, resulting in measuring 100 cells per well. Fiji software was used to quantify cell area and cell parameter, and then cell shape index was employed to quantify cell morphology.

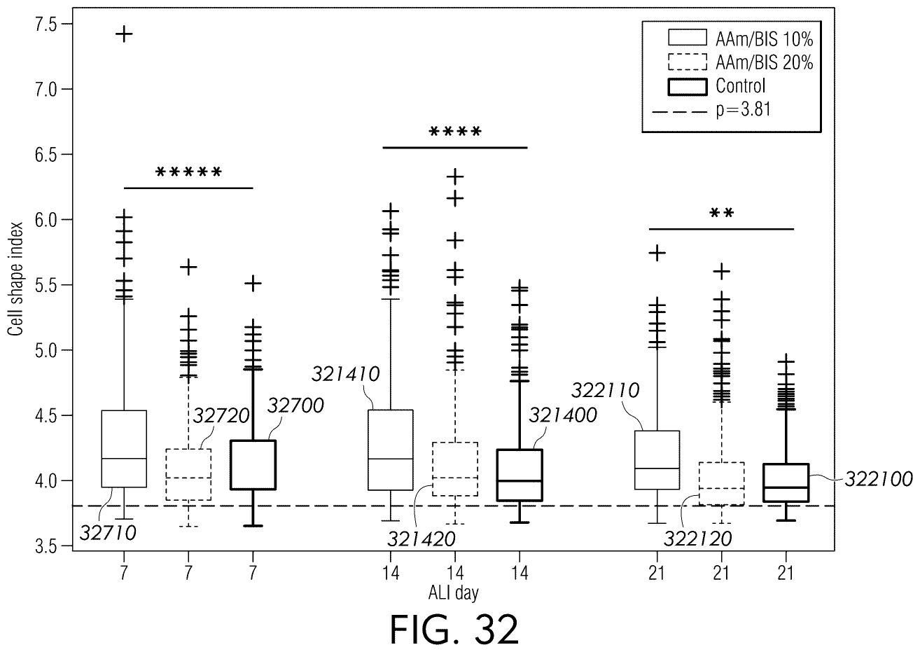

Quantitatively, cells on AAm/BIS 10% hydrogel had larger cell shape index with a median value of 4.2 on ALI day 7, as shown in FIG. 32, whereas the cell shape index of cells on AAm/BIS 20% gel was 4, indicating soft substrate stiffness might affect cell shape and stability. In FIG. 32, 32710 represents the results for a polyacrylamide hydrogel layer with AAm/BIS 10% for ALI day 7, 32720 represents the results for a polyacrylamide hydrogel layer with AAm/BIS 20% for ALI day 7, 32700 represents the results for no polyacrylamide hydrogel layer for ALI day 7, 321410 represents the results for a polyacrylamide hydrogel layer with AAm/BIS 10% for ALI day 14, 321420 represents the results for a polyacrylamide hydrogel layer with AAm/BIS 20% for ALI day 14, 321400 represents the results for no polyacrylamide hydrogel layer for ALI day 14, 322110 represents the results for a polyacrylamide hydrogel layer with AAm/BIS 10% for ALI day 21, 322120 represents the results for a polyacrylamide hydrogel layer with AAm/BIS 20% for ALI day 21, and 322100 represents the results for no polyacrylamide hydrogel layer for ALI day 21. After three weeks of ALI culture, the cell shape index showed no significant difference between various substrate stiffness, which suggests the maturity of the HBE cells. Moreover, the cell shape index decreased from ALI day 7 to day 21, confirming that mature cells have round or symmetric shapes. These results demonstrate that substrate stiffness affects the structure of HBE cells under ALI culture.

### Example 9: HBEC Culture-Cell Area

To examine the morphological changes of HBE cells under different substrate stiffnesses, the cell area was quantified on ALI days 7, 14, and 21, as shown in FIG. 24. Quantitatively, cells grown on PAAm hydrogel had larger cell area (400 μm²/cell on AAm/BIS 20% gel) compared to cells grown directly on plastic membrane on ALI day 21 (210 μm²/cell). For cells in the gel-ALI system, cells on AAm/BIS 10% gel had slightly larger cell area with a median value of 430 μm²/cell. These results indicate that substrate stiffness is associated with cell area and cell growth. Yet, the increased cell area on soft substrate slows down cell proliferation. Nevertheless, the cells on PAAm hydrogel have the capacity to differentiate into ciliated cells and goblet cells.

### Example 10: Electrical Resistance of the Porous Membrane

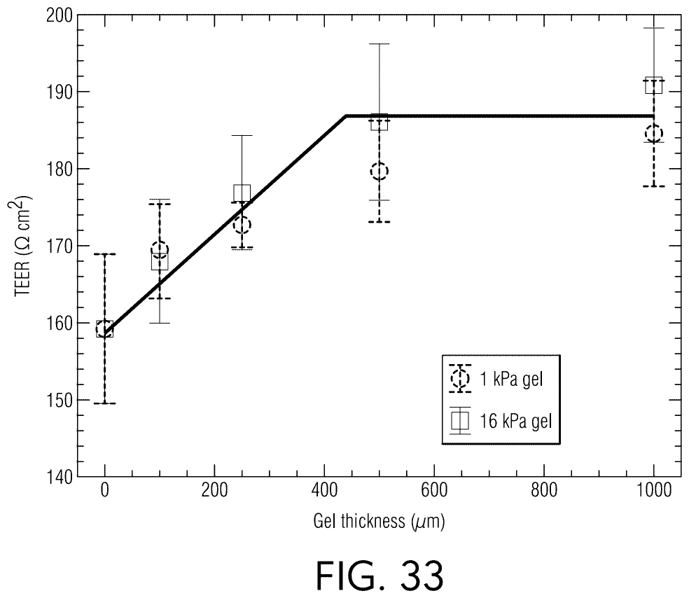

The effects of the polyacrylamide hydrogel layer on the electrical resistance of the porous plastic membrane were quantified. The electrical resistance reflects the ionic conductivity across the polyacrylamide hydrogel layer; therefore, the electrical resistance provides an indirect assessment of the transport properties of the air-liquid-interface apparatus. First, the electrical resistance of the semipermeable porous support was determined to have a value of 159±10 52 cm2. When the gel had a thickness of 100 μm, the increase in electric resistance was very small (approximately 5%) regardless of the polyacrylamide hydrogel stiffness, as shown in FIG. 33. Therefore, the 100 μm thick polyacrylamide hydrogel layer is thin enough for efficient nutrient transport.

What is claimed is:

1. An air-liquid-interface culture apparatus comprising:
a porous support comprising a polymer plate having a plurality of pores and a support surface; and
a polyacrylamide hydrogel layer deposited onto the support surface of the porous support, the polyacrylamide hydrogel layer comprising a polyacrylamide hydrogel.

2. The air-liquid-interface culture apparatus of claim 1, wherein the polyacrylamide hydrogel layer has a thickness of greater than or equal to 100 μm.

3. The air-liquid-interface culture apparatus of claim 1, wherein the polyacrylamide hydrogel layer has a Young's modulus of from 80 Pa to 21 kPa.

4. The air-liquid-interface culture apparatus of claim 1, wherein the polyacrylamide hydrogel layer has a mesh size of from 2 nm to 50 nm.

5. The air-liquid-interface culture apparatus of claim 1, further comprising one or more cells on the polyacrylamide hydrogel layer.

6. The air-liquid-interface culture apparatus of claim 1, further comprising a coating, wherein the coating is formed on an outer surface of the polyacrylamide hydrogel layer.

7. The air-liquid-interface culture apparatus of claim 6, wherein the coating comprises photoactivable heterobifunctional crosslinker, collagen, or combinations thereof.

8. The air-liquid-interface culture apparatus of claim 1, wherein the porous support comprises a surface area of from 0.05% to 1% pores and an average pore size of from 0.1 μm to 10 μm.

9. The air-liquid-interface culture apparatus of claim 1, wherein the polyacrylamide hydrogel is a reaction product resulting from polymerization of a hydrogel precursor solution comprising acrylamide monomer, N,N′-methylene bis-acrylamide crosslinker, ammonium persulfate initiator, and N,N,N′,N′-tetramethylethylenediamide initiator.

10. The air-liquid-interface culture apparatus of claim 9, wherein the hydrogel precursor solution comprises:
from 0.5 wt. % to 25 wt. % acrylamide monomer;
from 0.05 wt. % to 1 wt. % N,N′-methylene bis-acrylamide crosslinker;
from 0.0005 wt. % to 1 wt. % ammonium persulfate catalyst; and
from 0.005 wt. % to 1 wt. % N,N,N′,N′-tetramethylethylenediamide initiator.

11. A system for culturing cells, the system comprising:
the air-liquid-interface culture apparatus of claim 1, wherein the porous support further comprises one or more support arms; and
a nutrient medium, wherein the nutrient medium is held by a vessel;
wherein the one or more support arms are configured to support the polymer plate above a bottom of the vessel to form a lower nutrient compartment and an upper nutrient compartment.

12. A method of preparing an air-liquid-interface culture apparatus, the method comprising:
preparing a polyacrylamide hydrogel precursor solution wherein the polyacrylamide hydrogel precursor solution comprises acrylamide monomer, N,N′-methylene bis-acrylamide crosslinker, ammonium persulfate initiator, and N,N,N′,N′-tetramethylethylenediamide initiator;
pouring the polyacrylamide hydrogel precursor solution on to a porous surface; and
polymerizing the polyacrylamide hydrogel precursor solution to form a polyacrylamide hydrogel.

13. The method of claim 12, further comprising placing a coverslip over the polyacrylamide hydrogel precursor solution after pouring the polyacrylamide hydrogel precursor solution.

14. The method of claim 13, wherein a surface of the coverslip is hydrophobic.

15. The method of claim 12, wherein polymerizing the polyacrylamide hydrogel precursor solution occurs by free radical polymerization.

16. The method of claim 12, wherein the polyacrylamide hydrogel precursor solution comprises, based on the total weight of the polyacrylamide hydrogel precursor solution:
from 0.5 wt. % to 25 wt. % acrylamide monomer;
from 0.05 wt. % to 1 wt. % N,N′-methylene bis-acrylamide crosslinker;
from 0.0005 wt. % to 1 wt. % ammonium persulfate catalyst; and
from 0.005 wt. % to 1 wt. % N,N,N′,N′-tetramethylethylenediamide initiator.

17. The method of claim 12, wherein the polyacrylamide hydrogel precursor solution comprises a weight ratio of weight of the acrylamide monomer to the weight of the N,N′-methylenebisacrylamide of from 40:1 to 5:1.

18. The method of claim 12, further comprising:
applying sulfosuccinimidyl-6-(4′-azido-2′-nitrophenylamino) hexanoate to the polyacrylamide hydrogel to produce an activated outer surface of the polyacrylamide hydrogel; and
applying collagen to the activated outer surface of the polyacrylamide hydrogel.

19. A method of culturing cells, the method comprising:
applying cells to an air-liquid-interface culture apparatus, the air-liquid-interface culture apparatus comprising:
a porous support comprising a polymer plate having a plurality of pores and a support surface; and
a polyacrylamide hydrogel layer deposited onto the support surface of the porous support, the polyacrylamide hydrogel layer comprising a polyacrylamide hydrogel;
wherein the cells are applied to an upper surface of the polyacrylamide hydrogel layer; and
submerging the air-liquid-interface culture apparatus in a nutrient medium, wherein the nutrient medium is present below the porous support and the nutrient medium is also present above the polyacrylamide hydrogel layer;
incubating the cells until the cells reach at least 90% confluence; and
removing the nutrient medium from above the polyacrylamide hydrogel layer to form an air-liquid-interface.

20. The method of claim 19, further comprising:
activating an outer surface of the polyacrylamide hydrogel layer by applying photoactivable heterobifunctional crosslinker to the outer surface and exposing the photoactivable heterobifunctional crosslinker to ultra violet light; and
applying collagen to the outer surface of the polyacrylamide hydrogel layer.
